SEQUENCE LISTING (29 sequences)

SEQ ID NO: 1 - protein, 30 aa, synthetic construct
  VARIANT 1: note = X at position 1 is Phe, Gln or Ala
  VARIANT 2: note = X at position 2 is Val or Gly
  VARIANT 3: note = X at position 3 is Asn, Lys, Asp, Gly, Gln, Ala or Glu
  VARIANT 16: note = X at position 16 is Glu, Tyr, Gln or His
  VARIANT 25: note = X at position 25 is His or Phe
  VARIANT 27: note = X at position 27 is Gly, Thr, Ser, His, Val, or is absent
  VARIANT 28: note = X at position 28 is Gly, Glu, Pro, Lys, Asp, Ser, His, or is absent
  VARIANT 29: note = X at position 29 is Gly, Glu, Lys, Pro, Gln, Asp, His, or is absent
  VARIANT 30: note = X at position 30 is Gly, Thr, Ser, Glu, Lys, Ala, or is absent
XXXQHLCGSHLVEALXLVCGERGFXYXXXX

SEQ ID NO: 2 - protein, 22 aa, synthetic construct
  VARIANT 10: note = X at position 10 is Thr or Ile
  VARIANT 14: note = X at position 14 is Asp, Tyr, Gln or Glu
  VARIANT 21: note = X at position 21 is Gly, Asn, Ser, or Ala
  VARIANT 22: note = X at position 22 is any naturally occurring amino acid, or is absent
GIVEQCCTSXCSLXQLENYCXX

SEQ ID NO: 3 - protein, 7 aa, synthetic construct
  VARIANT 1: note = X at position 1 is Gly or is absent
  VARIANT 3: note = X at position 3 is Gly, Ser, or is absent
XGXGGGG

SEQ ID NO: 4 - protein, 59 aa, synthetic construct
  VARIANT 1: note = X at position 1 is Phe, Gln, or Ala
  VARIANT 2: note = X at position 2 is Val or Gly
  VARIANT 3: note = X at position 3 is Asn, Lys, Asp, Gly, Gln, Ala, or Glu
  VARIANT 16: note = X at position 16 is Glu, Tyr, Gln, or His
  VARIANT 25: note = X at position 25 is His or Phe
  VARIANT 27: note = X at position 27 is Gly, Thr, Ser, His, Val, or is absent
  VARIANT 28: note = X at position 28 is Gly, Glu, Pro, Lys, Asp, Ser, His, or is absent
  VARIANT 29: note = X at position 29 is Gly, Glu, Lys, Pro, Gln, Asp, His, or is absent
  VARIANT 30: note = X at position 30 is Gly, Thr, Ser, Glu, Lys, Ala, or is absent
  VARIANT 31: note = X at position 31 is Gly or is absent
  VARIANT 33: note = X at position 33 is Gly, Ser, or is absent
  VARIANT 47: note = X at position 47 is Thr or Ile
  VARIANT 51: note = X at position 51 is Asp, Tyr, Gln, or Glu
  VARIANT 58: note = X at position 58 is Gly, Asn, Ser, or Ala
  VARIANT 59: note = X at position 59 is any naturally occurring amino acid, or is absent
XXXQHLCGSHLVEALXLVCGERGFXYXXXXXGXGGGGGIVEQCCTSXCSLXQLENYCXX

SEQ ID NO: 5 - protein, 58 aa, synthetic construct
FVNQHLCGSHLVEALELVCGERGFHYGGGGGGSGGGGGIVEQCCTSTCSLDQLENYCG

SEQ ID NO: 6 - protein, 22 aa, synthetic construct
  VARIANT 5: note = X at position 5 is Gln or Glu
  VARIANT 10: note = X at position 10 is Gln or Glu
  VARIANT 15: note = X at position 15 is Gln or Glu
  VARIANT 20: note = X at position 20 is Gly, Glu, Gln, or is absent
  VARIANT 21: note = X at position 21 is Gly or absent
  VARIANT 22: note = X at position 22 is Gly or absent
GGGGXGGGGXGGGGXGGGGXXX

SEQ ID NO: 7 - protein, 20 aa, synthetic construct
GGGGQGGGGQGGGGQGGGGG

SEQ ID NO: 8 - protein, 222 aa, synthetic construct
  VARIANT 16: note = X at position 16 is Phe, Gln or Glu
  VARIANT 18: note = X at position 18 is Phe, Gln or Glu
  VARIANT 37: note = X at position 37 is Val or Thr
  VARIANT 39: note = X at position 39 is Val or Thr
  VARIANT 72: note = X at position 72 is Asn, Asp or Gln
  VARIANT 222: note = X at position 222 is Lys or is absent
CPPCPAPELLGGPSVXLXPPKPKDTLMISRTPEVTCXVXDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 9 - protein, 223 aa, synthetic construct
  VARIANT 17: note = X at position 17 is Phe, Gln or Glu
  VARIANT 19: note = X at position 19 is Phe, Gln or Glu
  VARIANT 38: note = X at position 38 is Val or Thr
  VARIANT 40: note = X at position 40 is Val or Thr
  VARIANT 73: note = X at position 73 is Asn, Asp or Gln
  VARIANT 185: note = X at position 185 is Arg or Lys
  VARIANT 223: note = X at position 223 is Lys or is absent
PCPPCPAPEAAGGPSVXLXPPKPKDTLMISRTPEVTCXVXDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSXLTVDKSRWQEGNVFSCSVMHEALHNHYTQKSLSLSLGX

SEQ ID NO: 10 - protein, 222 aa, synthetic construct
  VARIANT 16: note = X at position 16 is Phe, Gln, or Glu
  VARIANT 18: note = X at position 18 is Phe, Gln, or Glu
  VARIANT 37: note = X at position 37 is Val or Thr
  VARIANT 39: note = X at position 39 is Val or Thr
  VARIANT 72: note = X at position 72 is Asn, Asp, or Gln
  VARIANT 222: note = X at position 222 is Lys or is absent
ECPPCPAPPVAGPSVXLXPPKPKDTLMISRTPEVTCXVXDVSHEDPEVQFNWYVDGVEVHNAKTKPREEQFXSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGX

SEQ ID NO: 11 - protein, 303 aa, synthetic construct
  VARIANT 1: note = X at position 1 is Phe, Gln, or Ala
  VARIANT 2: note = X at position 2 is Val or Gly
  VARIANT 3: note = X at position 3 is Asn, Lys, Asp, Gly, Gln, Ala, or Glu
  VARIANT 16: note = X at position 16 is Glu, Tyr, Gln, or His
  VARIANT 25: note = X at position 25 is His or Phe
  VARIANT 27: note = X at position 27 is Gly, Thr, Ser, His, Val, or is absent
  VARIANT 28: note = X at position 28 is Gly, Glu, Pro, Lys, Asp, Ser, His, or is absent
  VARIANT 29: note = X at position 29 is Gly, Glu, Lys, Pro, Gln, Asp, His, or is absent
  VARIANT 30: note = X at position 30 is Gly, Thr, Ser, Glu, Lys, Ala, or is absent
  VARIANT 31: note = X at position 31 is Gly, or is absent
  VARIANT 33: note = X at position 33 is Gly, Ser, or is absent
  VARIANT 47: note = X at position 47 is Thr or Ile
  VARIANT 51: note = X at position 51 is Asp, Tyr, Gln, or Glu
  VARIANT 58: note = X at position 58 is Gly, Asn, Ser or Ala
  VARIANT 59: note = X at position 59 is any naturally occurring amino acid, or is absent
  VARIANT 64: note = X at position 64 is Gln or Glu
  VARIANT 69: note = X at position 69 is Gln or Glu
  VARIANT 74: note = X at position 74 is Gln or Glu
  VARIANT 79: note = X at position 79 is Gly, Glu, Gln, or is absent
  VARIANT 80: note = X at position 80 is Gly or is absent
  VARIANT 81: note = X at position 81 is Gly or is absent
  VARIANT 82: note = X at position 82 is Glu or Pro
  VARIANT 90: note = X at position 90 is Glu or Pro
  VARIANT 91: note = X at position 91 is Ala and Val
  VARIANT 94: note = X at position 94 is Gly or is absent
  VARIANT 125: note = X at position 125 is Gln or His
  VARIANT 157: note = X at position 157 is Tyr or Phe
  VARIANT 166: note = X at position 166 is Leu or Val
  VARIANT 187: note = X at position 187 is Ser or Ala
  VARIANT 188: note = X at position 188 is Ser or Pro
  VARIANT 196: note = X at position 196 is Ala or Thr
  VARIANT 212: note = X at position 212 is Gln or Arg
  VARIANT 254: note = X at position 254 is Val or Met
  VARIANT 266: note = X at position 266 is Arg or Lys
  VARIANT 276: note = X at position 276 is Glu or Gln
  VARIANT 302: note = X at position 302 is Leu or Pro
XXXQHLCGSHLVEALXLVCGERGFXYXXXXXGXGGGGGIVEQCCTSXCSLXQLENYCXXGGGGXGGGGXGGGGXGGGGXXXXCPPCPAPXXAGXPSVFLFPPKPKDTLMISRTPEVTCVVVDVSXEDPEVQFNWYVDGVEVHNAKTKPREEQFNSTXRVVSVLTVXHQDWLNGKEYKCKVSNKGLPXXIEKTISKXKGQPREPQVYTLPPSXEEMTKNQVSLTCLVKGFYPSDIAVEWESNGQPENNYKTTPPXLDSDGSFFLYSXLTVDKSRWQXGNVFSCSVMHEALHNHYTQKSLSLSXG

SEQ ID NO: 12 - protein, 299 aa, synthetic construct
FVNQHLCGSHLVEALELVCGERGFHYGGGGGGSGGGGGIVEQCCTSTCSLDQLENYCGGGGGQGGGGQGGGGQGGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 13 - protein, 21 aa, Homo sapiens
GIVEQCCTSICSLYQLENYCN

SEQ ID NO: 14 - protein, 30 aa, Homo sapiens
FVNQHLCGSHLVEALYLVCGERGFFYTPKT

SEQ ID NO: 15 - protein, 7 aa, synthetic construct
GGSGGGG

SEQ ID NO: 16 - protein, 300 aa, synthetic construct
FVNQHLCGSHLVEALYLVCGERGFFYTEETGGGGGGGIVEQCCTSICSLYQLENYCGGGGGSGGGGSGGGGSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 17 - protein, 302 aa, synthetic construct
FVNQHLCGSHLVEALELVCGERGFHYGGGGGGSGGGGGIVEQCCTSICSLDQLENYCGGGGGQGGGGQGGGGQGGGGQGGPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 297 aa, synthetic construct
FVNQHLCGSHLVEALELVCGERGFHYGGGGGSGGGGGIVEQCCTSICSLDQLENYCGGGGGEGGGGEGGGGEGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 19 - protein, 302 aa, synthetic construct
FVNQHLCGSHLVEALELVCGERGFHYGGGGGGSGGGGGIVEQCCTSTCSLDQLENYCGGGGGQGGGGQGGGGQGGGGQGGPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 302 aa, synthetic construct
FVGQHLCGSHLVEALELVCGERGFHYGGGGGGSGGGGGIVEQCCTSICSLDQLENYCGGGGGQGGGGQGGGGQGGGGQGGPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 21 - protein, 297 aa, synthetic construct
AGGQHLCGSHLVEALELVCGERGFHYGGGGGSGGGGGIVEQCCTSICSLDQLENYCGGGGGQGGGGQGGGGQGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 22 - protein, 299 aa, synthetic construct
FVNQHLCGSHLVEALELVCGERGFHYTPKTGGSGGGGGIVEQCCTSTCSLDQLENYCGGGGGQGGGGQGGGGQGGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 23 - protein, 298 aa, synthetic construct
FVNQHLCGSHLVEALELVCGERGFFYTEETGGGGGGGIVEQCCTSICSLYQLENYCGGGGGSGGGGSGGGGSGGGGSECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 24 - protein, 299 aa, synthetic construct
FVGQHLCGSHLVEALELVCGERGFHYGGGGGGSGGGGGIVEQCCTSTCSLDQLENYCGGGGGQGGGGQGGGGQGGGGGECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 25 - protein, 31 aa, Homo sapiens
HAEGTFTSDVSSYLEGQAAKEFIAWLVKGRG

SEQ ID NO: 26 - protein, 5 aa, synthetic construct
  VARIANT 5: note = X at position 5 is Gln, Glu, or Ser
GGGGX

SEQ ID NO: 27 - protein, 6 aa, Homo sapiens
ERKCCV

SEQ ID NO: 28 - protein, 6 aa, Homo sapiens
ESKYGP

SEQ ID NO: 29 - protein, 10 aa, Homo sapiens
EPKSCDKTHT

(19) United States
(12) Patent Application Publication
(10) Pub. No.: US 2025/0032588 A1
(43) Pub. Date: Jan. 30, 2025

(54) FUSION PROTEINS

(71) Applicant: ELI LILLY AND COMPANY, Indianapolis, IN (US)

(72) Inventors: David Bruce BALDWIN, Carmel, IN (US); John Michael BEALS, Solana Beach, CA (US); Jonathan Wesley DAY, Carmel, IN (US); Craig Duane DICKINSON, San Diego, CA (US); Andrew Ihor KORYTKO, Oceanside, CA (US); Gregory Alan LAZAR, Pacifica, CA (US)

(21) Appl. No.: 18/756,062

(22) Filed: Jun. 27, 2024

Related U.S. Application Data
(62) Division of application No. 17/577,932, filed on Jan. 18, 2022, now Pat. No. 12,059,452.
Application No. 17/577,932 is a division of application No. 16/893,498, filed on Jun. 5, 2020, now Pat. No. 11,253,574.
Application No. 16/893,498 is a division of application No. 15/820,608, filed on Nov. 22, 2017, now Pat. No. 10,709,766.
Application No. 15/820,608 is a division of application No. 15/140,519, filed on Apr. 28, 2016, now Pat. No. 9,855,318.
(60) Provisional application No. 62/158,079, filed on May 7, 2015.

(51) Int. Cl. A61K 38/28; A61K 38/00; A61K 38/16; A61K 38/26; C07K 14/00; C07K 14/62

(52) U.S. Cl. CPC A61K 38/28; A61K 38/16; A61K 38/26; C07K 14/001; C07K 14/62; A61K 38/00; C07K 2319/30

(57) ABSTRACT

The present invention relates to fusion proteins comprising an insulin receptor agonist fused to a human IgG Fc region through the use of a peptide linker, and the use of such fusion proteins in the treatment of diabetes. The fusion protein of the present invention has an extended time action profile and is useful for providing basal glucose control for an extended period of time.

The present application is being filed along with a Sequence Listing. The Sequence Listing is provided as a file titled “X20356D_US.xml” created Oct. 8, 2024, and is 66,681 bytes in size. The Sequence Listing is incorporated herein by reference in its entirety.

The present invention relates to fusion proteins for use in the treatment of diabetes. More particularly, the invention relates to fusion proteins comprising an insulin receptor agonist fused to a human IgG Fc region with a peptide linker, and the use of such proteins in the treatment of diabetes. The fusion proteins of the present invention have an extended time action profile and are useful for providing protracted basal glucose control and suppression of hepatic glucose output.

Diabetes mellitus is a chronic disorder characterized by hyperglycemia resulting from defects in insulin secretion, insulin action, or both. Type 1 diabetes mellitus is characterized by little or no insulin secretory capacity, and patients with type 1 diabetes mellitus require insulin for survival. Type 2 diabetes mellitus is characterized by elevated blood glucose levels resulting from impaired insulin secretion, insulin resistance, excessive hepatic glucose output, and/or contributions from all of the above. In at least one-third of patients with Type 2 diabetes mellitus, the disease progresses to an absolute requirement for insulin therapy.

In order to achieve normal glycemia, insulin replacement therapy is desired to parallel, as closely as possible, the pattern of endogenous insulin secretion in normal individuals. The daily physiological demand for insulin fluctuates and can be separated into two phases: (a) the mealtime phase requiring a pulse (bolus) of insulin to dispose of the meal-related blood glucose surge, and (b) the inter-meal phase requiring a sustained (basal) amount of insulin to regulate hepatic glucose output for maintaining optimal fasting blood glucose.

Because Type 1 diabetes patients produce little or no insulin, effective insulin therapy for Type 1 diabetics generally involves the use of two types of exogenously administered insulin: a rapid-acting, mealtime insulin provided by bolus injections, and a long-acting, basal insulin, administered once or twice daily to control blood glucose levels between meals. Treatment of patients with Type 2 diabetes typically begins with prescribed weight loss, exercise, and a diabetic diet, but when these measures fail to control elevated blood sugars, then oral medications and incretin-based therapy, such as administration of glucagon-like peptide-1 (GLP-1) receptor agonists and/or dipeptidyl peptidase 4 (DPP-4) inhibitors that enable increased incretin levels, may be necessary. When these medications are still insufficient, treatment with insulin is considered. Type 2 diabetes patients whose disease has progressed to the point that insulin therapy is required are generally started on a single daily injection of a long-acting, basal insulin, although mealtime injections of rapid-acting insulins may be included, as necessary, in some cases.

Several types of basal insulins are currently available. Insulin glargine, sold under the tradename LANTUS®, comprises a modified insulin structure in which the asparagine at position 21 in the insulin A-chain is replaced with glycine, and two arginines are added to the C-terminus of the B-chain. Insulin detemir, sold under the tradename LEVEMIR®, comprises a modified insulin structure in which the threonine at position 30 of the B-chain has been deleted and the lysine at position 29 of the B-chain has been derivatized through the covalent linkage of a 14-carbon, myristoyl fatty acid to the s-amine group of lysine at B29. Insulin degludec, available in Europe and Japan under the tradename TRESIBA®, comprises a modified insulin structure in which the threonine at position 30 of the B-chain has been deleted, and the ε-amino group of the lysine at position 29 of the B-chain is covalently derivatized with hexadecandioic acid via a γ-L-glutamic acid linker. All of these insulins are indicated for once-daily administration.

Treatment regimens involving daily injections of existing insulin therapies can be complicated and painful to administer and can result in undesired side effects, such as hypoglycemia and weight gain. Therefore, many diabetic patients are unwilling or unable to comply, or are incapable of complying, with the insulin therapy necessary to maintain close control of blood glucose levels. Poor glycemic control increases a patient's risk for developing serious diabetes-related complications, including heart disease, stroke, nerve damage, lower limb amputation, vision loss, and kidney disease. Research is being conducted to identify insulin products with longer duration of action; thus, requiring fewer injections than currently available insulin products to improve acceptance and compliance.

CN103509118 describes proteins with a human insulin B-chain and human insulin A-chain joined by a 4 to 50 amino acid C-peptide connection sequence, and with the insulin A-chain attached directly, without an additional linker, to an immunoglobulin Fc fragment, and states that testing in mice shows that such proteins have an in vivo half-life as long as about 3 days. KR101324828 describes proinsulin analogs linked to immunoglobulin Fc regions through the use of non-peptidyl linkers, and states that such proteins provide increased serum half-life over existing therapies. The publication states that the non-peptidyl linkers represent an improvement over peptide linkers, asserting that fusion proteins using peptide linkers cannot increase the half-life of an active medication in the blood because peptide linkers are easily severed by enzymes in the body.

Despite the disclosures above, and/or in any other publications, the present inventors overcame multiple obstacles to discover fusion proteins comprising insulin receptor agonists, peptide linkers, and human IgG Fc regions that meet the ongoing need for a glucose-lowering product with increased duration of action, sufficient for less frequent dosing than currently available insulin products, including dosing as infrequently as once-weekly. For example, in order to achieve the desired prolonged time action profile, it was necessary to engineer fusion proteins with attenuated potency to avoid rapid receptor mediated clearance, a major route of insulin clearance, but that still have enough potency to provide sufficient glucose lowering. Further, in order to minimize renal clearance, the other major route of insulin clearance, and to regulate peripheral exposure through hydrodynamic size-limited paracellular diffusion, fusion proteins had to be engineered which were sufficiently large, and which would not have the insulin receptor agonist proteolytically cleaved from the human IgG Fc region after being administered, as such cleavage would result in faster than desired renal clearance of the insulin receptor agonist even if the human IgG Fc region remained in circulation. In addition, IgG Fc domains have evolved to self-associate to form stable dimers, and when such a dimer is formed from two fusion proteins, each comprising an insulin moiety fused with an IgG Fc region, the two insulin moieties are brought into close proximity to one another, enabling self-association, or dimerization, of the insulin moieties, mediated through the insulin B-chain self-association regions. Such insulin dimers are inactive, so fusion proteins with reduced self-association of the insulin receptor agonist moieties had to be engineered. Multiple additional challenges were overcome to create a fusion protein suitable for commercial manufacture and formulation as a therapeutic. The present invention provides fusion proteins which have prolonged duration of action compared to existing insulin treatments, allowing for less frequent injections than existing insulin products, including up to once weekly, thus reducing the complexity of and pain associated with existing treatment regimens involving more frequent injections. The fusion proteins of the present invention have a flat pharmacokinetic profile and restricted peripheral exposure, resulting in low day-to-day variability, and minimal incidence of hypoglycemia, including when provided in combination with an additional diabetes medication, such as an incretin-based therapy. The fusion proteins of the present invention may also provide prolonged duration of action without causing weight gain.

The present invention provides a fusion protein comprising:
a) an insulin receptor agonist having the general formula Z₁-Z₂-Z₃, wherein
   i) Z₁ is an insulin B-chain analog, comprising the amino acid sequence:

X₁X₂X₃QHLCGSHLVEALX₄LVCGERGFX₅YX₆X₇X₈X₉

wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X₉ is G, T, S, E, K, A or is absent, provided that the insulin B-chain analog includes at least one modification from the amino acid sequence of the B-chain of a molecule of human insulin at X₄, X₅, X₆, X₇, X₈, or X₉ (SEQ ID NO:1);
   ii) Z₂ is a first peptide linker comprising 5 to 10 amino acids, wherein at least 5 of said amino acids are G residues; and
   iii) Z₃ is an insulin A-chain analog comprising the amino acid sequence:

GIVEQCCTSX₁CSLX₂QLENYCX₃X₄

X₁ is T or I; X₂ is D, Y, Q or E; X₃ is G, N, S or A; and X₄ is any naturally occurring amino acid, or is absent, provided that if X₃ is N, then X₄ must be an amino acid other than Gor N (SEQ ID NO:2);
b) a second peptide linker; and
c) a human IgG Fc region;
   wherein the C-terminal residue of the insulin receptor agonist is directly fused to the N-terminal residue of the second peptide linker, and the C-terminal residue of the second peptide linker is directly fused to the N-terminal residue of the IgG Fc region.

The present invention also provides a fusion protein consisting of:
a) an insulin receptor agonist having the general formula Z₁-Z₂-Z₃, wherein
   i) Z₁ is an insulin B-chain analog, having the amino acid sequence:

X₁X₂X₃QHLCGSHLVEALX₄LVCGERGFX₅YX₆X₇X₈X₉

wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X₉ is G, T, S, E, K, A or is absent, provided that the insulin B-chain analog includes at least one modification from the amino acid sequence of the B-chain of a molecule of human insulin at X₄, Xs, X₆, X₇, X₈, or X₉ (SEQ ID NO:1);
   ii) Z₂ is a first peptide linker comprising 5 to 10 amino acids, wherein at least 5 of said amino acids are G residues; and
   iii) Z₃ is an insulin A-chain analog having the amino acid sequence:

GIVEQCCTSX₁CSLX₂QLENYCX₃X₄

X₁ is T or I; X₂ is D, Y, Q or E; X₃ is G, N, S or A; and X₄ is any naturally occurring amino acid, or is absent, provided that if X₃ is N, then X₄ must be an amino acid other than Gor N (SEQ ID NO:2);
b) a second peptide linker; and
c) a human IgG Fc region;
   wherein the C-terminal residue of the insulin receptor agonist is directly fused to the N-terminal residue of the second peptide linker, and the C-terminal residue of the second peptide linker is directly fused to the N-terminal residue of the IgG Fc region.

The present invention also provides a pharmaceutical composition comprising a fusion protein of the present invention and at least one pharmaceutically acceptable excipient.

The present invention also provides a method of treating a patient with diabetes mellitus, obesity, dyslipidemia or metabolic syndrome, comprising administering to a patient in need thereof a therapeutically effective amount of a fusion protein of the present invention. The present invention also provides a method of treating or preventing a diabetes-related condition selected from the group consisting of heart disease, stroke, nephropathy, retinopathy, and kidney disease, comprising administering to a patient in need thereof a therapeutically effective amount of a fusion protein of the present invention.

The invention also provides a fusion protein of the present invention for use in therapy.

The present invention also provides the use of a fusion protein of the present invention in the manufacture of a medicament.

The present invention also provides polynucleotides encoding a fusion protein of the present invention.

The present invention also provides a process for producing a fusion protein of the present invention, said process comprising the steps of:
1. culturing a mammalian host cell comprising a polynucleotide encoding a fusion protein of the present invention under conditions such that said fusion protein is expressed; and
2. recovering from said host cell a fusion protein;

The present invention also provides a fusion protein produced by the process described above.

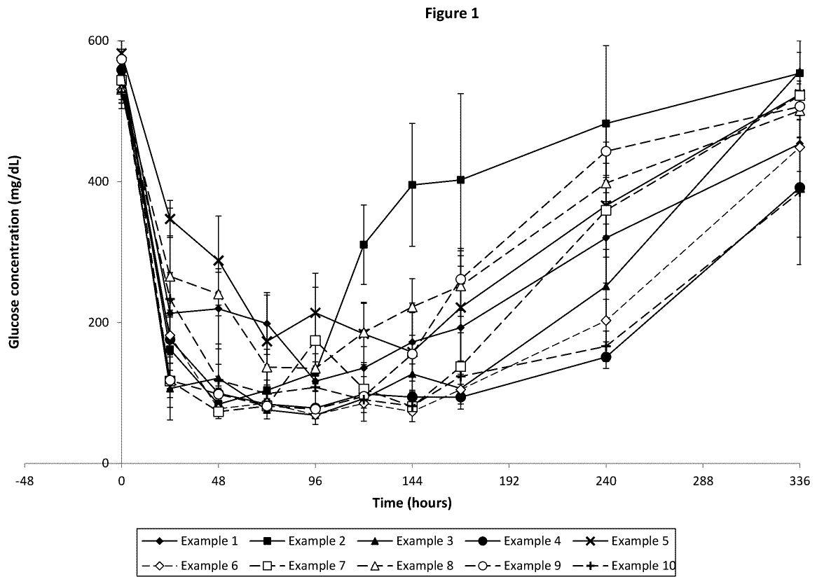

FIG. 1. FIG. 1 provides pharmacodynamic data for exemplary fusion proteins of the present invention in a streptozotocin (STZ)-treated rat diabetes model.

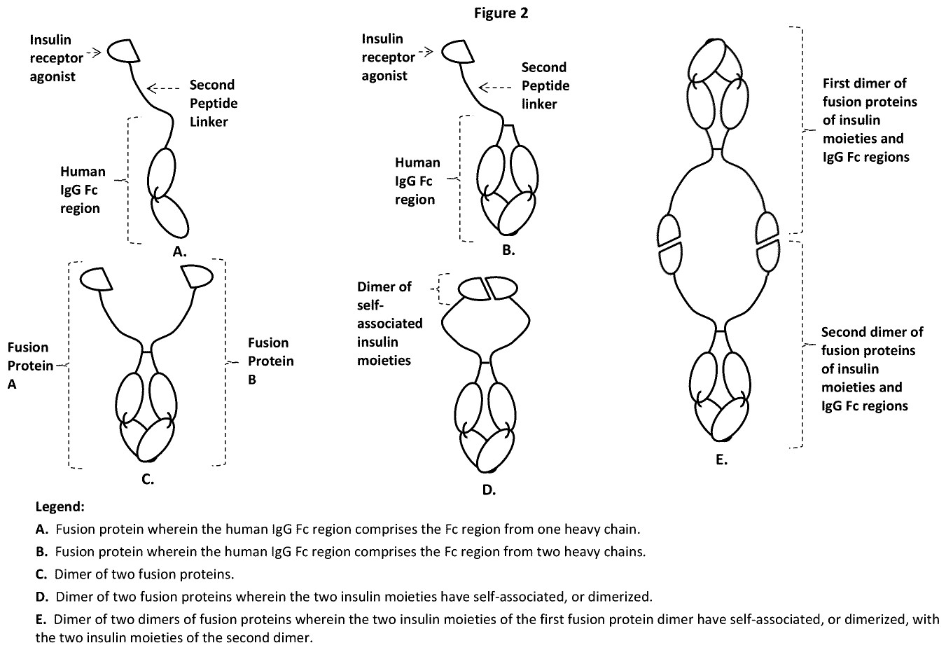

FIG. 2. FIG. 2 provides a schematic diagram of configurations of proteins described herein. It should be noted that the particular shapes (e.g., ovals, half-circles, etc.) used in the diagrams in FIG. 2 are not intended to describe or characterize, and should not be used to construe, the meaning or structure of the individual components of the fusion proteins of the present invention.

In certain embodiments, the insulin B-chain analog includes at least one modification from the amino acid sequence of the B-chain of a molecule of human insulin at X₄ or X₅ of SEQ ID NO:1, and at least one modification from the amino acid sequence of the B-chain of a molecule of human insulin at X₆, X₇, X₈, or X₉ of SEQ ID NO:1.

In certain embodiments, the insulin B-chain analog includes at least two modifications from the amino acid sequence of the B-chain of a molecule of human insulin at X₆, X₇, X₈, or X₉ of SEQ ID NO:1.

In certain embodiments, the insulin B-chain analog includes modifications from the amino acid sequence of the B-chain of a molecule of human insulin at X₆, X₇, X₈, and X₉ of SEQ ID NO:1.

In certain embodiments, the insulin B-chain analog has the amino acid sequence of SEQ ID NO: 1 wherein X₆-X₉ are each G.

In certain embodiments, the insulin B-chain analog includes modifications from the amino acid sequence of the B-chain of a molecule of human insulin at X₄ and X₅ of SEQ ID NO:1.

In certain embodiments, the insulin B-chain analog has the sequence of SEQ ID NO:1, wherein: X₁ is F; X₂ is V; X₃ is N or D; X₄ is E; X₅ is H.

In certain embodiments, the insulin B-chain analog includes modification from the amino acid sequence of the B-chain of a molecule of human insulin at each of positions X₄, X₅, X₆, X₇, X₈, and X₉ of SEQ ID NO:1.

In certain embodiments, the insulin B-chain analog comprises the sequence of SEQ ID NO:1, wherein: X₁ is F; X₂ is V; X₃ is N or D; X₄ is E; X₅ is H; and X₆-X₉ are each G.

In certain embodiments, the insulin A-chain analog includes at least one modification from the amino acid sequence of the human insulin A-chain at X₁ or X₂ of SEQ ID NO:2.

In certain embodiments, the insulin A-chain analog includes modifications from the amino acid sequence of the human insulin A-chain at both X₁ and X₂ of SEQ ID NO:2.

In certain embodiments, the insulin A-chain analog has the sequence of SEQ ID NO:2, wherein: X₁ is I or T; X₂ is D; X₃ is G; and X₄ is absent.

In certain embodiments, the insulin A-chain analog has the sequence of SEQ ID NO:2, wherein X₃ is N and wherein X₄ is an amino acid other than G, N, S, V L or P.

In certain embodiments, the insulin B-chain analog includes at least one modification from the amino acid sequence of the B-chain of a molecule of human insulin at X₄ or X₅ of SEQ ID NO:1, and at least one modification from the amino acid sequence of the B-chain of a molecule of human insulin at X₆, X₇, X₈, or X₉ of SEQ ID NO:1; and the insulin A-chain analog includes at least one modification from the amino acid sequence of the human insulin A-chain at X₁ or X₂ of SEQ ID NO:2.

In certain embodiments, the insulin B-chain analog includes at least two modifications from the amino acid sequence of the B-chain of a molecule of human insulin at X₆, X₇, X₈, or X₉ of SEQ ID NO:1; and the insulin A-chain analog includes at least one modification from the amino acid sequence of the human insulin A-chain at X₁ or X₂ of SEQ ID NO:2.

In certain embodiments, the insulin B-chain analog includes modifications from the amino acid sequence of the B-chain of a molecule of human insulin at X₆, X₇, X₈, and X₉ of SEQ ID NO:1; and the insulin A-chain analog includes at least one modification from the amino acid sequence of the human insulin A-chain at X₁ or X₂ of SEQ ID NO:2.

In certain embodiments, the insulin B-chain analog has the amino acid sequence of SEQ ID NO:1 wherein X₆-X₉ are each G; and the insulin A-chain analog includes at least one modification from the amino acid sequence of the human insulin A-chain at X₁ or X₂ of SEQ ID NO:2.

In certain embodiments, the insulin B-chain analog includes modifications from the amino acid sequence of the B-chain of a molecule of human insulin at X₄ and X₅ of SEQ ID NO:1; and the insulin A-chain analog includes at least one modification from the amino acid sequence of the human insulin A-chain at X₁ or X₂ of SEQ ID NO:2.

In certain embodiments, the insulin B-chain analog comprises the sequence of SEQ ID NO:1, wherein: X₁ is F; X₂ is V; X₃ is N or D; X₄ is E; X₅ is H; and the insulin A-chain analog includes at least one modification from the amino acid sequence of the human insulin A-chain at X₁ or X₂ of SEQ ID NO:2.

In certain embodiments, the insulin B-chain analog includes modification from the amino acid sequence of the B-chain of a molecule of human insulin at each of positions X₄, X₅, X₆, X₇, X₈, and X₉ of SEQ ID NO:1; and the insulin A-chain analog includes at least one modification from the amino acid sequence of the human insulin A-chain at X₁ or X₂ of SEQ ID NO:2.

In certain embodiments, the insulin B-chain analog has the sequence of SEQ ID NO:1, wherein: X₁ is F; X₂ is V; X₃ is N or D; X₄ is E; X₅ is H; and X₆-X₉ are each G; and the insulin A-chain analog includes at least one modification from the amino acid sequence of the human insulin A-chain at X₁ or X₂ of SEQ ID NO:2.

In certain embodiments, the insulin B-chain analog includes at least one modification from the amino acid sequence of the B-chain of a molecule of human insulin at X₄ or X₅ of SEQ ID NO:1, and at least one modification from the amino acid sequence of the B-chain of a molecule of human insulin at X₆, X₇, X₈, or X₉ of SEQ ID NO:1; and the insulin A-chain analog includes modifications from the amino acid sequence of the human insulin A-chain at both X₁ and X₂ of SEQ ID NO:2.

In certain embodiments, the insulin B-chain analog includes at least two modifications from the amino acid sequence of the B-chain of a molecule of human insulin at X₆, X₇, X₈, or X₉ of SEQ ID NO:1; and the insulin A-chain analog includes modifications from the amino acid sequence of the human insulin A-chain at both X₁ and X₂ of SEQ ID NO:2.

In certain embodiments, the insulin B-chain analog includes modifications from the amino acid sequence of the B-chain of a molecule of human insulin at X₆, X₇, X₈, and X₉ of SEQ ID NO:1; and the insulin A-chain analog includes modifications from the amino acid sequence of the human insulin A-chain at both X₁ and X₂ of SEQ ID NO:2.

In certain embodiments, the insulin B-chain analog has the amino acid sequence of SEQ ID NO:1 wherein X₆-X₉ are each G; and the insulin A-chain analog includes modifications from the amino acid sequence of the human insulin A-chain at both X₁ and X₂ of SEQ ID NO:2.

In certain embodiments, the insulin B-chain analog includes modifications from the amino acid sequence of the B-chain of a molecule of human insulin at X₄ and X₅ of SEQ ID NO:1; and the insulin A-chain analog includes modifications from the amino acid sequence of the human insulin A-chain at both X₁ and X₂ of SEQ ID NO:2.

In certain embodiments, the insulin B-chain analog comprises the sequence of SEQ ID NO:1, wherein: X₁ is F; X₂ is V; X₃ is N or D; X₄ is E; X₅ is H; and the insulin A-chain analog includes modifications from the amino acid sequence of the human insulin A-chain at both X₁ and X₂ of SEQ ID NO:2.

In certain embodiments, the insulin B-chain analog includes modification from the amino acid sequence of the B-chain of a molecule of human insulin at each of positions X₄, X₅, X₆, X₇, X₈, and X₉ of SEQ ID NO:1; and the insulin A-chain analog includes modifications from the amino acid sequence of the human insulin A-chain at both X₁ and X₂ of SEQ ID NO:2.

In certain embodiments, the insulin B-chain analog has the sequence of SEQ ID NO:1, wherein: X₁ is F; X₂ is V; X₃ is N or D; X₄ is E; X₅ is H; and X₆-X₉ are each G; and the insulin A-chain analog includes modifications from the amino acid sequence of the human insulin A-chain at both X₁ and X₂ of SEQ ID NO:2.

In certain embodiments, the insulin B-chain analog includes at least one modification from the amino acid sequence of the B-chain of a molecule of human insulin at X₄ or X₅ of SEQ ID NO:1, and at least one modification from the amino acid sequence of the B-chain of a molecule of human insulin at X₆, X₇, X₈, or X₉ of SEQ ID NO:1; and the insulin A-chain analog has the sequence of SEQ ID NO:2, wherein: X₁ is I or T; X₂ is D; X₃ is G; and X₄ is absent.

In certain embodiments, the insulin B-chain analog includes at least two modifications from the amino acid sequence of the B-chain of a molecule of human insulin at X₆, X₇, X₈, or X₉ of SEQ ID NO:1; and the insulin A-chain analog has the sequence of SEQ ID NO:2, wherein: X₁ is I or T; X₂ is D; X₃ is G; and X₄ is absent.

In certain embodiments, the insulin B-chain analog includes modifications from the amino acid sequence of the B-chain of a molecule of human insulin at X₆, X₇, X₈, and X₉ of SEQ ID NO:1; and the insulin A-chain analog has the sequence of SEQ ID NO:2, wherein: X₁ is I or T; X₂ is D; X₃ is G; and X₄ is absent.

In certain embodiments, the insulin B-chain analog has the amino acid sequence of SEQ ID NO:1 wherein X₆-X₉ are each G, wherein: X₁ is I or T; X₂ is D; X₃ is G; and X₄ is absent.

In certain embodiments, the insulin B-chain analog includes modifications from the amino acid sequence of the B-chain of a molecule of human insulin at X₄ and X₅ of SEQ ID NO:1; and the insulin A-chain analog has the sequence of SEQ ID NO:2, wherein: X₁ is I or T; X₂ is D; X₃ is G; and X₄ is absent.

In certain embodiments, the insulin B-chain analog comprises the sequence of SEQ ID NO:1, wherein: X₁ is F; X₂ is V; X₃ is N or D; X₄ is E; X₅ is H; and the insulin A-chain analog has the sequence of SEQ ID NO:2, wherein: X₁ is I or T; X₂ is D; X₃ is G; and X₄ is absent.

In certain embodiments, the insulin B-chain analog includes modification from the amino acid sequence of the B-chain of a molecule of human insulin at each of positions X₄, X₅, X₆, X₇, X₈, and X₉ of SEQ ID NO:1; and the insulin A-chain analog has the sequence of SEQ ID NO:2, wherein: X₁ is I or T; X₂ is D; X₃ is G; and X₄ is absent.

In certain embodiments, the insulin B-chain analog has the sequence of SEQ ID NO:1, wherein: X₁ is F; X₂ is V; X₃ is N or D; X₄ is E; X₅ is H; and X₆-X₉ are each G; and the insulin A-chain analog has the sequence of SEQ ID NO:2, wherein: X₁ is I or T; X₂ is D; X₃ is G; and X₄ is absent.

In certain embodiments, the first peptide linker (Z₂) comprises the amino acid sequence: X₁GX₂GGGG, wherein X₁ is G or is absent; and X₂ is G, S or is absent (SEQ ID NO:3).

In certain embodiments, the insulin receptor agonist, i.e., Z₁-Z₂-Z₃ in the fusion protein described above, comprises the amino acid sequence:

X₁X₂X₃QHLCGSHLVEALX₄LVCGERGFX₅YX₆X₇X₈X₉X₁₀GX₁₁GGGG
GIVEQCCTSX₁₂CSLX₁₃QLENYCX₁₄X₁₅

wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X₉ is G, T, S, E, K, A or is absent; X₁₀ is G or is absent; X₁₁ is G, S or is absent; X₁₂ is T or I; X₁₃ is D, Y, Q or E; X₁₄ is G, N, S or A; and X₁₅ is any naturally occurring amino acid, or is absent, provided that at least one of X₄, Xs, X₆, X₇, X₈, or X₉ must be a different amino acid than that found, respectively, at position B₁₆, B₂₅, B₂₇, B₂₈, B₂₉ or B₃₀ of the B-chain of a molecule of human insulin, and further provided that if X₁₄ is N, then X₁₅ must be an amino acid other than Gor N (SEQ ID NO:4).

In certain preferred embodiments, the insulin receptor agonist, i.e., Z₁-Z₂-Z₃ in the fusion protein described above has the following amino acid sequence:

(SEQ ID NO: 5)
FVNQHLCGSHLVEALELVCGERGFHYGGGGGGSGGGGGIVEQCCTSTCSL
DQLENYCG.

In certain embodiments, the second peptide linker is a peptide having between 10 and 25 amino acids, wherein at least 50% of said amino acids are G residues. In certain embodiments, the second peptide linker is a peptide comprising the amino acid sequence [GGGGX]ₙ, wherein X is Q, E or S; and wherein n is 2-5 (SEQ ID NO:26). In certain embodiments, the second peptide linker comprises the amino acid sequence:

GGGGX₁GGGGX₂GGGGX₃GGGGX₄X₅X₆

wherein X₁ is Q or E; X₂ is Q or E; X₃ is Q or E; X₄ is G, E, Q or is absent; X₅ is G or absent; and X₆ is G or is absent (SEQ ID NO:6).

In certain preferred embodiments, the second peptide linker has the amino acid sequence:

(SEQ ID NO: 7)
GGGGQGGGGQGGGGQGGGGG.

In certain embodiments, the human IgG Fc region comprises fragments from one heavy chain of an IgG antibody. A schematic diagram of a fusion protein comprising such an IgG region is provided in diagram (A) in FIG. 2. In other embodiments, the human IgG Fc region comprises fragments from two heavy chains of an IgG antibody. A schematic diagram of a fusion protein comprising such an IgG region is provided in diagram (B) in FIG. 2.

In certain embodiments, the human IgG Fc region is an Fc region from an IgG1, IgG2 or IgG4 antibody.

In certain embodiments, the human IgG Fc region is an Fc region from an IgG1 antibody comprising the following amino acid sequence:

CPPCPAPELLGGPSVX₁LX₂PPKPKDTLMISRTPEVTCX₃VX₄DVSHEDP
EVKFNWYVDGVEVHNAKTKPREEQYX₅STYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGX₆

wherein X₁ is F, Q or E; X₂ is F, Q or E; X₃ is V or T; X₄ is V or T; X₅ is N, D or Q; and X₆ is K or is absent. (SEQ ID NO:8)

In certain embodiments, the IgG Fc region comprises the amino acid sequence of SEQ ID NO:8 and further comprises some or all of the amino acids that would be found in a wild-type IgG1 Fc sequence to the N-terminal side of the C residue at position 1 in SEQ ID NO:8.

Preferably, the human IgG Fc region is from either an IgG2 or IgG4 antibody.

In certain embodiments, the human IgG Fc region is an Fc region from an IgG4 antibody comprising the following amino acid sequence:

PCPPCPAPEAAGGPSVX₁LX₂PPKPKDTLMISRTPEVTCX₃VX₄DVSQED
PEVQFNWYVDGVEVHNAKTKPREEQFX₅STYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSX₆LTVDKSRWQ
EGNVFSCSVMHEALHNHYTQKSLSLSLGX₇

wherein X₁ is F, Q or E; X₂ F, Q or E; X₃ is V or T; X₄ is V or T; X₅ is N, D or Q; X₆ is R, or K; X₇ is K or is absent. (SEQ ID NO:9)

In certain embodiments, the IgG Fc region comprises the amino acid sequence of SEQ ID NO:9, and further comprises some or all of the amino acids that would be found in a wild type IgG4 Fc sequence to the N-terminal side of the C residue at position 1 in SEQ ID NO:9. In certain embodiments, the human IgG Fc region comprises the amino acid sequence of SEQ ID NO:9, wherein X₁ is F; X₂ is F; X₃ is V; X₄ is V; X₅ is N; X₆ is R; and X₇ is absent.

In certain embodiments, the human IgG Fc region is an Fc region from an IgG2antibody having the following amino acid sequence:

ECPPCPAPPVAGPSVX₁LX₂PPKPKDTLMISRTPEVTCX₃VX₄DVSHEDP
EVQFNWYVDGVEVHNAKTKPREEQFX₅STFRVVSVLTVVHQDWLNGKEYK
CKVSNKGLPAPIEKTISKTKGQPREPQVYTLPPSREEMTKNQVSLTCLVK
GFYPSDIAVEWESNGQPENNYKTTPPMLDSDGSFFLYSKLTVDKSRWQQG
NVFSCSVMHEALHNHYTQKSLSLSPGX₆

wherein X₁ is F, Q or E; X₂ is F, Q or E; X₃ is V or T; X₄ is V or T; X₅ is N, D or Q; and X₆ is K or absent. (SEQ ID NO:10)

In certain embodiments, the IgG Fc region comprises the amino acid sequence of SEQ ID NO:10, and further comprises some or all of the amino acids that would be found in a wild type IgG2 Fc sequence to the N-terminal side of the E residue at position 1 in SEQ ID NO:10. In certain embodiments, the human IgG Fc region comprises the amino acid sequence of SEQ ID NO:10, wherein X₁ is F; X₂ is F; X₃ is V; X₄ is V; X₅ is N; and X₆ is absent.

In certain embodiments, the fusion protein comprises the amino acid sequence:

X₁X₂X₃QHLCGSHLVEALX₄LVCGERGFX₅YX₆X₇X₈X₉X₁₀GX₁₁GGGG
GIVEQCCTSX₁₂CSLX₁₃QLENYCX₁₄X₁₅GGGGX₁₆GGGGX₁₇GGGG
X₁₈GGGGX₁₉X₂₀X₂₁X₂₂CPPCPAPX₂₃X₂₄AGX₂₅PSVFLFPPKPKDT
LMISRTPEVTCVVVDVSX₂₆EDPEVQFNWYVDGVEVHNAKTKPREEQFNS
TX₂₇RVVSVLTVX₂₈HQDWLNGKEYKCKVSNKGLPX₂₉X₃₀IEKTISK
X₃₁KGQPREPQVYTLPPSX₃₂EEMTKNQVSLTCLVKGFYPSDIAVEWESN
GQPENNYKTTPX₃₃LDSDGSFFLYSX₃₄LTVDKSRWQX₃₅GNVFSCSVMH
EALHNHYTQKSLSLSX₃₆G

wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X₉ is G, T, S, E, K, A or is absent, provided that at least one of X₄, X₅, X₆, X₇, X₈, or X₉ is an amino acid other than that which is present, respectively, at position B₁₆, B₂₅, B₂₇, B₂₈, B₂₉ or B₃₀ of a human insulin B-chain; X₁₀ is G or is absent; X₁₁ is G, S or is absent; X₁₂ is T or I; X₁₃ is D, Y, Q or E; X₁₄ is G, N, S or A; X₁₅ is any naturally occurring amino acid, or is absent, provided that if X₁₄ is N, then X₁₅ must be an amino acid other than G or N; X₁₆ is Q or E; X₁₇ is Q or E; X₁₈ is Q or E; X₁₉ is G, E, Q or is absent; X₂₀ is G or absent; X₂₁ is G or is absent; X₂₂ is E or P; X₂₃ is E or P; X₂₄ is A or V; X₂₅ is G or is absent; X₂₆ is Q or H; X₂₇ is Y or F; X₂₈ is L or V; X₂₉ is S or A; X₃₀ is S or P; X₃₁ is A or T; X₃₂ is Q or R; X₃₃ is V or M; X₃₄ is R or K; X₃₅ is E or Q; and X₃₆ is L or P (SEQ ID NO:11).

In certain embodiments, the present invention provides a fusion protein selected from the group consisting of SEQ ID NO:12, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24.

In a preferred embodiment, the fusion protein has the amino acid sequence:

```
(SEQ ID NO: 12)
                  10 20 30 40 50 60
FVNQHLCGSHLVEALELVCGERGFHYGGGGGGSGGGGGIVEQCCTSTCSLDQLENYCGGG
                  70 80 90 100 110 120
GGQGGGGQGGGGQGGGGGECPPCPAPPVAGPSVFLFPPKPKDTLMISRTPEVTCVVVDVS
                  130 140 150 160 170 180
HEDPEVQFNWYVDGVEVHNAKTKPREEQFNSTFRVVSVLTVVHQDWLNGKEYKCKVSNKG
                  190 200 210 220 230 240
LPAPIEKTISKTKGQPREPQVYTLPPSREEMTKNQVSLTCLVKGFYPSDIAVEWESNGQP
                  250 260 270 280 290
ENNYKTTPPMLDSDGSFFLYSKLTVDKSRWQQGNVFSCSVMHEALHNHYTQKSLSLSPG.
```

In certain embodiments, the fusion proteins of the present invention are present in the form of a dimer. A schematic diagram of such a dimer is provided in diagram (C) in FIG. 2. In certain embodiments, the dimer is a homodimer, wherein the amino acid sequences of the two fusion proteins that make the dimer are the same. In certain embodiments, the dimer is a heterodimer, wherein the amino acid sequences of the two fusion proteins that make up the dimer are different.

In certain embodiments, the pharmaceutical composition of the present invention comprises a fusion protein of the present invention, a buffering agent, a surfactant, and an isotonicity agent. In certain embodiments, the buffering agent is citric acid and/or citrate, the surfactant is polysorbate 80, and the isotonicity agent is mannitol. In certain embodiments, the pH of the composition ranges from about 5.5 to about 8.0. In certain embodiments, the pH ranges from about 6.0 to about 7.4. In certain embodiments, the pH ranges from about 6.0 to 6.75.

In certain embodiments, the pharmaceutical composition further comprises an additional active ingredient. In certain embodiments, the additional active ingredient is an incretin-based therapy. In certain preferred embodiments, the incretin-based therapy is a GLP-1R agonist. Preferably, the GLP-1R agonist is dulaglutide.

In certain embodiments, the method of the present invention comprises administration of a therapeutically effective amount of a fusion protein once daily. In preferred embodiments, a therapeutically effective amount of the fusion protein is administered once weekly. In certain embodiments, a therapeutically effective amount of the fusion protein is administered once monthly. In certain embodiments, the present invention provides a method of treating a patient with diabetes mellitus while reducing the risk of hypoglycemia and/or weight gain, comprising administering to the patient a therapeutically effective amount of a fusion protein of the present invention.

The present invention also provides a method of treating a patient with diabetes mellitus, obesity, dyslipidemia, and/or metabolic syndrome, comprising administering a therapeutically effective amount of a fusion protein of the present invention in combination with an additional active ingredient. The fusion protein and additional active ingredient in such embodiments may be administered simultaneously, sequentially or in a single combined formulation. In certain embodiments, the additional active ingredient is an incretin-based therapy. In certain preferred embodiments, the incretin-based therapy is a GLP-1R agonist. Preferably, the GLP-1R agonist is dulaglutide. In certain embodiments, the combination is administered once daily. In certain preferred embodiments, the combination is administered once weekly. In certain embodiments, the combination is administered once monthly.

In certain embodiments, the present invention provides a fusion protein of the present invention for use in treatment of diabetes mellitus, obesity, dyslipidemia or metabolic syndrome. In certain embodiments, the present invention provides a fusion protein of the present invention for use in treating or preventing a diabetes-related condition selected from the group consisting of heart disease, stroke, nephropathy, retinopathy, and kidney disease. In certain embodiments, the present invention provides a fusion protein of the present invention for use in treating a patient with diabetes mellitus while reducing the risk of hypoglycemia and/or weight gain, comprising administering to the patient a fusion protein of the present invention. In certain embodiments, the fusion protein of the present invention is provided for use in simultaneous, separate or sequential combination with another active ingredient. In certain embodiments, the additional active ingredient is an incretin-based therapy. In certain preferred embodiments, the incretin-based therapy is a GLP-1R agonist.

In certain embodiments, the present invention provides the use of a fusion protein of the present invention in the manufacture of a medicament for the treatment of diabetes mellitus, obesity, dyslipidemia or metabolic syndrome. In certain embodiments, the present invention provides the use of a fusion protein of the present invention in the manufacture of a medicament for the treatment or prevention of a diabetes-related condition selected from the group consisting of heart disease, stroke, nephropathy, retinopathy, and kidney disease. In certain embodiments, the present invention provides the use of a fusion protein of the present invention in the manufacture of a medicament for treating diabetes mellitus while reducing the risk of hypoglycemia and/or weight gain, comprising administering to the patient a fusion protein of the present invention. In certain embodiments, the present invention provides the use of a fusion protein of the present invention in the manufacture of a medicament for the treatment of diabetes mellitus, obesity, dyslipidemia or metabolic syndrome, wherein the medicament is to be administered simultaneously, separately or sequentilaly in combination with another active ingredient.

When used herein, the term “insulin receptor agonist” refers to a protein that binds to and activates the insulin receptor, resulting in a lowering of blood glucose levels and/or suppression of hepatic glucose output, characteristics which can be tested and measured using known techniques, such as those shown in the studies described below.

The insulin receptor agonist portion of the fusion proteins of the present invention includes an analog of an insulin B-chain and an analog of an insulin A-chain. When used herein, the terms “insulin A-chain” and “insulin B-chain” refer to the A and B chains of the human insulin molecule (CAS No. 11061-68-0), whose native wild type sequences are well-known. The human insulin A-chain consists of 21 amino acids, referred to in the art as A₁-A₂₁, having the following sequence:

(SEQ ID NO: 13)
GIVEQCCTSICSLYQLENYCN.

The human insulin B-chain consists of 30 amino acids, referred to in the art as B₁-B₃₀, having the following sequence:

(SEQ ID NO: 14)
FVNQHLCGSHLVEALYLVCGERGFFYTPKT.

In a molecule of human insulin, the A-and B-chains are joined by two disulfide bonds, CysA7-CysB₇ and CysA20-CysB₁₉. The A-chain has an intra-chain disulfide bond at CysA6-CysA11.

To achieve the desired extended time action profile, the insulin receptor agonist of the fusion protein of the present invention must remain in circulation, and be capable of interacting with the insulin receptor, over an extended period of time. In order for the fusion proteins of the present invention to remain in circulation for the desired period of time, elimination of the fusion proteins must be attenuated. The two primary routes of insulin elimination are renal clearance and insulin receptor-mediated clearance. See Iglesias P, et al. Diabetes Obes. Metab. 2008; 10:811-823. To minimize renal clearance, a molecule with hydrodynamic size of at least about the size of human serum albumin is needed, and such a hydrodynamic size is provided in the fusions proteins of the present invention by the human IgG Fc region. As for receptor-mediated clearance, the fusion protein cannot be so potent at the insulin receptor that it results in more rapid receptor-mediated clearance than desired, but the fusion protein must be potent enough, however, to provide sufficient glucose control at doses that are commercially feasible. Thus, the potency of the fusion protein must be carefully balanced, and the structure of the fusion protein of the present invention allows it to achieve such a balance.

Insulin molecules have a tendency to self-associate into dimers and hexamers. Numerous roles have been proposed for the evolutionarily-conserved, self-association tendencies of insulin, including: (1) chemical and thermal stabilization of the molecule during intracellular vacuole storage; (2) protection of the monomeric insulin from fibrillation in vivo; (3) substitution for chaperone-assisted stabilization and folding during intracellular expression; and/or (4) essential for secretory trafficking. The active form of insulin, however, is the monomer.

The tendency of insulin molecules to self-associate, and the inactivity of such self-associated molecules, is relevant to the present invention, because human IgG Fc regions also tend to self-associate to form dimers, typically associated covalently through disulfide bonds in the hinge region, and such dimers are formed from the human IgG Fc regions in the fusion proteins of the present invention. As a result of the dimerization of the human IgG Fc regions, the two insulin receptor agonist “arms” are in close proximity to one another, and thereby exist at relatively high local concentration. In the case of human insulin, such close proximity would tend to favor self-association, or dimerization, of the insulin moieties, affecting the activity of the molecules. A schematic diagram of an Fc fusion protein dimer with insulin moiety arms that have become self-associated, or dimerized, is provided in diagram (D) of FIG. 2. The tendency of insulin molecules to self-associate could also lead to self-association, or dimerization, of insulin moieties from more than one Fc fusion protein dimer; a schematic diagram of a dimer of two Fc fusion protein dimers with self-associated, or dimerized, insulin moieties is provided in diagram (E) of FIG. 2. Further, the insulin moieties in more than two Fc fusion protein dimers could also self-associate in such a fashion to form, for example, a trimer comprised of three dimers, or even higher order aggregates comprised of more than three dimers.

The insulin receptor agonist portion of the fusion protein of the present invention, however, has a reduced tendency to self-associate or dimerize, and thus fusion protein dimers comprised of fusion proteins of the present invention tend to favor the structure depicted in diagram (C) of FIG. 2, as opposed to the structures depicted in diagrams (D) and (E) of FIG. 2. Thus, while the present invention provides a dimer of two fusion proteins, the insulin receptor agonist “arm” of each fusion protein in the dimer maintains a predominately monomeric state, as depicted for example in diagram (C) of FIG. 1, and is thus more capable of interacting with the insulin receptor.

In the fusion proteins of the present invention, the analog of the insulin B-chain in the insulin receptor agonist includes one or more modifications to the amino acid sequence of the human insulin B-chain. In particular, in order to reduce the propensity of the insulin receptor agonist portions to self-associate, or dimerize, the insulin B-chain analog includes one or more modifications from the B-chain of a molecule of human insulin at positions B₁₆, B₂₅ or B₂₇-30, which are represented in SEQ ID NO:1 as positions X₄, X₅ and X₆-9, respectively. For example: X₄ (which corresponds with B₁₆ in the B-chain of a human insulin molecule) may be modified to E, Q or H; X₅ (which corresponds with B₂₅ in the B-chain of a human insulin molecule) may be modified to H; X₆ (which corresponds with B₂₇ in the B-chain of a human insulin molecule) may be deleted or modified to G, S, H or V; X₇ (which corresponds with B₂₈ in the B-chain of a human insulin molecule) may be deleted or modified to G, E, K, D, S or H; X₈ (which corresponds with B₂₉ in the B-chain of a human insulin molecule) may be deleted or modified to G, E, P, Q, D or H; and X₉ (which corresponds with B₃₀ in the B-chain of a human insulin molecule) may be deleted or modified to G, S, E or K. In addition to reducing the propensity of the insulin receptor agonist portions to self-associate, modifications to positions B₁₆ and B₂₅ on the insulin B-chain analog—X₄ and X₅ of SEQ ID NO:1, respectively—may also be made to adjust potency, improve expression, improve chemical and/or physical stability, improve the ease with which the fusion proteins can be formulated with other commonly used excipients and/or to eliminate deamidation. The insulin B-chain analog may also include additional modifications for these reasons. Referring to the variables in SEQ ID NO:1, such additional modifications include the following: modification of X₁ (which corresponds with B₁ in the B-chain of a human insulin molecule) to Q or A; modification of X₂ (which corresponds with B₂ in the B-chain of a human insulin molecule) to G; and/or modification of X₃ (which corresponds with B₃ in the B-chain of a human insulin molecule) to K, D or G.

In certain preferred embodiments, the insulin B-chain analog includes more than one modification to the amino acid sequence of the human insulin B-chain at positions X₄, X₅ and X₆-9 of SEQ ID NO:1. In a preferred embodiment, X₄ is E and X₅ is H. In certain embodiments, the amino acid sequence of X₆-X₉ of SEQ ID NO:1 is selected from the group consisting of: GGES, GGGS, GGDS, GGEG, GGGG, SSES, SSGS, GGEE, GGGE, GGEK, GGGK, TPGS, TGGS, HGES, GHES, GGHS, GGEH, HGGS, GHGS, GGGH, GGDD, VGES, TEET, TKPT, GGGG, TGGG, TPGG, EPKT, TDKT, TPGS, EGGS, EGES, EEES, EPES, EPEP and GGDD. In preferred embodiments, the sequence of these four amino acids is GGGS, GGGG or TEET. In particularly preferred embodiments, X₄ is E, X₅ is H and X₆₋₉ is GGGG.

It should be noted that, while X₆-X₉ of SEQ ID NO:1 are described above as comprising the C-terminal end of the insulin B-chain analog (Z₁), these amino acids are not critical to the activity of the fusion proteins at the insulin receptor, and thus may alternatively be considered an extension of the first peptide linker (Z₂). For example, in the context of SEQ ID NO:4, X₆-X₉ may be considered either part of the insulin B-chain analog or the first peptide linker in that insulin receptor agonist.

In the fusion proteins of the present invention, the analog of the insulin A-chain in the insulin receptor agonist portion may include one or more modifications to the amino acid sequence of the human insulin A-chain intended to improve chemical and physical stability, adjust potency, and/or enhance expression. Referring to the variables in SEQ ID NO:2, these modifications include the following: modification of X₁ (which corresponds with A₁₀ in the A-chain of a human insulin molecule) to T; modification of X₂ (which corresponds with A₁₄ in the A-chain of a human insulin molecule) to D, Q or E; and/or modification of X₃ (which corresponds with A₂₁ in the A-chain of a human insulin molecule) to G, S or A. In a preferred embodiment, X₁ is T, X₂ is D, and X₃ is G.

Further, in order to avoid deamidation as well as chemical and/or proteolytic cleavage, if the amino acid at position 21 in the analog of the insulin A-chain in the insulin receptor agonist, i.e., X₃ in SEQ ID NO:2, is an N—the amino acid that is present at the corresponding position in a molecule of human insulin-it must not be immediately followed at the C-terminal side by certain amino acids, such as a G or an N, or, in certain embodiments, a P, S, V or L. See, e.g., Vlasak J, Ionescu R., MAbs. 2011 May-Jun;3(3):253-63. Fragmentation of Monoclonal Antibodies. It should be noted that while this requirement is recited in the first fusion protein described above in the context of the options for positions X₄ and X₅ in SEQ ID NO:2, which pertains to the analog of the insulin A-chain, it is not critical for the non-glycine residue following an asparagine residue at position 21 in the analog of the insulin A-chain to be considered part of the insulin receptor agonist, as opposed to the second peptide linker. For example, in the context of SEQ ID NO:11, the residue corresponding with position 21 in the A-chain analog portion is represented by X₁₄, and the following amino acid, X₁₅, could either be considered part of the insulin receptor agonist or the second peptide linker.

As described above, in the fusion proteins of the present invention, the C-terminal residue of the insulin B-chain analog is directly fused to the N-terminal residue of a first peptide linker and the C-terminal residue of the first peptide linker is directly fused to the N-terminal residue of the insulin B-chain analog. The first peptide linker must provide sufficient flexibility for the analogs of the insulin A-chain and B-chain to achieve the structure necessary to bind to the insulin receptor, but must not be so long that it unduly interferes with that binding. The length and composition of the first peptide linker may be adjusted in order to adjust the potency and/or expression of the fusion proteins. In some embodiments, the first peptide linker is 5 to 10 amino acids in length, at least 5 of which are G residues. In certain embodiments, the amino acid sequence of the first peptide linker is selected from the group consisting of: GGGGGG, GGGGG, EGGGGG, GEGGGG, GGEGGG, GGGEGG, GGGGEG, GGGGGE, KGGGGG, GKGGGG, GGKGGG, GGGKGG, GGGGKG, GGGGGK, HGGGGG, GHGGGG, GGHGGG, GGGHGG, GGGGHG, GGGGGH, GGGGGA, GGGGGR, SGGGGG, GSGGGG, GGSGGG, GGSGGGK, GGSGGGG and GGSGGG. In certain preferred embodiments, the sequence of the first peptide linker comprises SEQ ID NO:3. Most preferably, the sequence of the first peptide linker is GGSGGGG (SEQ ID NO:15). The insulin receptor agonist portion of the fusion proteins of the present invention also includes the disulfide bonds found in a molecule of human insulin as described above, namely, two disulfide bonds joining the analogs of the insulin A-chain and B-chain at CysA₇-CysB₇ and CysA₂₀-CysB₁₉, and an intra-chain disulfide bond in the analog of the insulin A-chain at CysA₆-CysA11.

As described above, the C-terminal residue of the insulin receptor agonist portion of the fusion proteins of the present invention is fused to the N-terminal residue of a second peptide linker, and the C-terminal residue of the second peptide linker is fused directly to the N-terminal residue of the Fc portion. It is preferred that the second peptide linker be glycine rich, to provide sufficient conformational flexibility. Preferably, the second peptide linker is less than 30 amino acids in length. In certain preferred embodiments, the second peptide linker is between 10 and 25 amino acids in length, with at least 50% of the amino acids being glycine residues. A preferred second peptide linker includes the sequence (GGGGX₁₅) , wherein X₁₅ is Q, E or S and n=2-5 (SEQ ID NO:26). A more preferred second peptide linker has the amino acid sequence of SEQ ID NO:6. A most preferred second peptide linker has the amino acid sequence GGGGQGGGGQGGGGQGGGGG (SEQ ID NO:7).

As used herein, the term “human IgG Fc region” has the meaning commonly given to the term in the field of immunology. Specifically, this term refers to a human IgG antibody fragment which is obtained by removing the two antigen binding regions (the Fab fragments) from the antibody. In particular, the Fc region includes the CH2 and CH3 constant region domains of the antibody, and may also include some or all of the hinge region.

As described above, in certain embodiments of the fusion protein of the present invention, the human IgG Fc region comprises fragments of the constant region from one heavy chain of an IgG antibody, a schematic depiction of which is provided in diagram (A) of FIG. 2, and in other embodiments, the human IgG Fc region comprises fragments of the constant regions from two heavy chains of an IgG antibody, a schematic depiction of which is provided in diagram (B) of FIG. 2. In this embodiment, the constant regions of the two heavy chains are associated with one another through non-covalent interactions and disulfide bonds.

There are four IgG subclasses (G1, G2, G3, and G4) each of which has different structures and biological functions known as effector functions. These effector functions are generally mediated through interaction with the Fc receptor (FcγR) or by binding complement factor C1q. Binding to FcγR can lead to antibody-dependent cell-mediated cytolysis, whereas binding to complement factors can lead to complement-mediated cell lysis. The structures and properties of the Fc regions of the IgG subclasses are known in the art. The fusion proteins of the present invention may contain Fc regions from any of the IgG subclasses, although G2 and G4 have lower receptor binding and effector function activities than G1 and G3 antibodies, and are thus preferred.

When used herein, the term human IgG Fc region also includes versions of such antibody fragments which have been modified, elongated and/or truncated, for example, to alter properties or characteristics such as the complement and/or Fc receptor binding functions, effector functions, disulfide bond formation, glycosylation, antibody-dependent cell-mediated cytotoxicity (ADCC), manufacturability, and/or stability. For example, the human IgG Fc regions of the fusion proteins of the present invention may be modified to reduce or remove the N-linked glycosylation site, which will reduce C1q binding affinity and cytotoxicity, and which may aid in immunogenicity, affect conformational stability and in vivo clearance rate, and/or modify the effector functions. The human IgG Fc regions of the fusion proteins of the present invention may also have some or all of the hinge region removed in order to simplify disulfide mediated Fc dimerization. Other examples of alterations include phosphorylation, sulfation, acylation, glycosylation, methylation, acetylation, amidation, and/or modifications to enable production of heterodimer molecules. Techniques for modifying the structures and properties of the human IgG Fc regions of the IgG subclasses are known in the art.

Regardless of the final structure of the fusion protein, the human IgG Fc region must serve to prolong the in vivo plasma half-life of the insulin receptor agonist. In preparing heterologous Fc fusion proteins wherein the Fc portion is being utilized for its ability to extend half-life, it is important to minimize any effector function. Furthermore, the fused insulin receptor agonist must remain able to bind to and activate the insulin receptor to result in a lowering of blood glucose levels and/or suppression of hepatic glucose output, characteristics which can be tested and measured using known techniques, such as those shown in the studies described below. A long half-life in the fusion proteins of the present invention can be demonstrated using, for example, the methods described below.

One preferred human IgG Fc region is an IgG4 Fc region modified to further reduce effector function, promote homodimer formation, and having a portion of the hinge deleted, as in SEQ ID NO:9 wherein X₁ is F; X₂ is F; X₃ is V; X₄ is V; X₅ is N; X₆ is R; and X₇ is absent.

Another preferred human IgG Fc region is an IgG2 Fc region having a portion of the hinge deleted, as in SEQ ID NO:10 wherein X₁ is F; X₂ is F; X₃ is V; X₄ is V; X₅ is N; and X₆ is absent.

It should be noted that, although the amino acid sequences of the preferred human IgG Fc regions recited above have portions of the hinge regions removed to simplify disulfide mediated dimerization, those hinge regions may be present in certain embodiments. For example, a wild-type IgG2 Fc region includes the six amino acid sequence ERKCCV (SEQ ID NO:27) at its N-terminal end, and although these amino acids are not recited in the IgG2 Fc region sequence set forth in SEQ ID NO:10, it is contemplated that a human IgG Fc region which comprises the amino acid sequence set forth in SEQ ID NO:10 may further comprise some or all of the six amino acid sequence ERKCCV (SEQ ID NO:27) at its N-terminal end. Similarly, a human IgG Fc region which comprises the amino acid sequence set forth in SEQ ID NO:9 may further comprise some or all of the six amino acids found at the N-terminal end of an IgG4 Fc region, namely ESKYGP (SEQ ID NO:28). Likewise, a human IgG Fc region which comprises the amino acid sequence set forth in SEQ ID NO:8 may further comprise some or all of the ten amino acids found at the N-terminal end of an IgG1 Fc region, namely: EPKSCDKTHT (SEQ ID NO:29). Moreover, the precise delineation between which amino acid constitutes the C-terminal end of the second peptide linker and which amino acid constitutes the N-terminal end of the human IgG Fc region is not critical to the structure or function of the fusion protein of the present invention. For example, in the context of SEQ ID NO:11, the residues corresponding with positions X₁₉-X₂₂ could be described as either the C-terminal end of the second peptide linker or an N-terminal extension of the human IgG Fc region.

As described above, the present invention also relates to polynucleotides that encode any of the fusion proteins of the present invention. The polynucleotides encoding the above-described fusion proteins may be in the form of RNA or DNA, which includes cDNA and synthetic DNA, and which may be double-stranded or single-stranded. The coding sequences that encode the proteins of the present invention may vary as a result of the redundancy or degeneracy of the genetic code.

The polynucleotides that encode for the fusion proteins of the present invention may include the following: only the coding sequence for the proteins, the coding sequence for the proteins and additional coding sequence, such as a leader or secretory sequence or a pro-protein sequence; the coding sequence for the proteins and non-coding sequence, such as introns or non-coding sequence 5′ and/or 3′ of the coding sequence for the proteins. Thus, the term “polynucleotide encoding a protein” encompasses a polynucleotide that may include not only coding sequence for the proteins but also a polynucleotide that includes additional coding and/or non-coding sequence.

The polynucleotides of the present invention will be expressed in a host cell after the sequences have been operably linked to an expression control sequence. The expression vectors are typically replicable in the host organisms either as episomes or as an integral part of the host chromosomal DNA. Commonly, expression vectors will contain selection markers to permit detection of those cells transformed with the desired DNA sequences.

The fusion proteins of the present invention may readily be produced in mammalian cells such as CHO, NSO, HEK293, BHK, or COS cells; in bacterial cells such as E. coli, Bacillus subtilis, or Pseudomonas fluorescence; in insect cells, or in fungal or yeast cells, which are cultured using techniques known in the art. Insect, and yeast or other fungal cells, however, produce non-human N-glycans, so proteins with N-linked glycosylation produced in such cells may cause immunogenic reactions if administered to patients. Production in such cells thus requires elimination of N-linked glycosylation sites and/or genetic engineering of the cells to produce human N-glycans using techniques known in the art. See, e.g., Hamilton S R, et al., Production of complex human glycoproteins in yeast, 301 SCIENCE (5637): 1244-6 (August 2003). Production in mammalian cells is preferred, and the preferred mammalian host cell is the CHOK1SV cell line containing a glutamine synthetase (GS) expression system (see U.S. Pat. No. 5,122,464).

The vectors containing the polynucleotide sequences of interest (e.g., the fusion proteins and expression control sequences) can be transferred into the host cell by well-known methods, which vary depending on the type of cellular host. For example, calcium chloride transformation is commonly utilized for prokaryotic cells, whereas calcium phosphate treatment or electroporation may be used for other cellular hosts.

Various methods of protein purification may be employed and such methods are known in the art and described, for example, in Deutscher, Methods in Enzymology 182:83-89 (1990) and Scopes, Protein Purification: Principles and Practice, 3rd Edition, Springer, N.Y. (1994).

As described above, the fusion protein of the present invention in certain embodiments is produced as a dimer. In such a dimer, the human IgG Fc regions of the fusion proteins are associated with one another through non-covalent interactions and disulfide bonds. A schematic depiction of such a dimer is provided in diagram (C) of FIG. 2. When the amino acid sequences of the two fusion proteins that make up such a dimer—e.g., Fusion Protein A and Fusion Protein B in the dimer depicted in diagram (C) of FIG. 2—are the same, the dimer is referred to herein as a “homodimer.” As noted above, expression of fusion proteins of the present invention in mammalian cells is preferred, and expression in such cells results in homodimers. The fusion proteins in such homodimers are associated through non-covalent interactions and intermolecular disulfide bonds in the Fc portion. For example, the protein produced by a gene which encodes the fusion protein of SEQ ID NO:12 would be a homodimer covalently bonded through inter-chain disulfide bonds, namely C80 to C80 and C83 to C83.

When the amino acid sequences of two fusion proteins that make up a dimer—e.g., Fusion Protein A and Fusion Protein B in diagram (C) of FIG. 2—are different, the dimer is referred to herein as a “heterodimer.” Such a heterodimer may be prepared by techniques known in the art. See, e.g, Lewis S M, et al. NAT. BIOTECHNOL. 32(2): 191-8 (2014); Carter, J. IMMUNOL. METHODS, 248(1-2): 7-15 (2001); Ridgway, J. B. et al. PROTEIN ENG. 9(7):617-2(1996).

References herein to pharmaceutical compositions comprising a fusion protein include pharmaceutical compositions which contain a homodimer of that fusion protein, and/or which contain a heterodimer, wherein one member of the heterodimer is that fusion protein. Similarly, references herein to methods comprising administering a fusion protein, include methods comprising administering a homodimer of that fusion protein and/or administering a heterodimer, wherein one member of the heterodimer is that fusion protein. Likewise, references to a fusion protein for use in therapy and/or a fusion protein for use in the manufacture of a medicament include a homodimer of that fusion protein, and/or a heterodimer wherein one member of the heterodimer is that fusion protein, for use in therapy and/or in the manufacture of a medicament.

The term “treatment” or “treating,” as used herein, refers to the management and care of a patient having diabetes or hyperglycemia, or other condition for which insulin administration is indicated for the purpose of combating or alleviating symptoms and complications of those conditions. Treating includes administering fusion proteins of the present invention to prevent or delay the onset of symptoms or complications, alleviating the symptoms or complications, or eliminating the disease, condition, or disorder. The patient to be treated is a mammal, and preferably, a human being.

The term “prevent” or “preventing,” as used herein, refers to reducing the risk or incidence of, or eliminating or slowing the progression of, one or more conditions, symptoms, complications or disorders.

The fusion proteins of the present invention can be used to treat subjects with a wide variety of diseases and conditions. Included are subjects with hyperglycemia, insulin-dependent diabetes as well as subjects with non-insulin dependent diabetes, including treatment naïve subjects as well as subjects being treated with oral medications, such as a sulfonylurea, metformin, thiazolidinedione such as pioglitazone, α-glucosidase inhibitor such as acarbose, and/or noninsulin injectables, including incretin-based therapies, such as DPP-4 inhibitors and GLP-IR agonists. The fusion proteins of the present invention may be used to regulate blood glucose in such patients, and may treat conditions or complications that result from insufficient blood glucose control such as retinopathy, neuropathy or kidney disease.

In certain embodiments, the fusion protein of the present invention is administered every day, every other day, twice weekly, thrice weekly, once weekly, twice monthly or once monthly. In preferred embodiments, the duration of action is sufficiently extended to allow for once weekly dosing. Even for such long acting molecules, however, it will be recognized by those of skill in the art that effective glucose control may also be provided by gradually dose accumulating a drug with a long pharmacokinetic profile using a more frequent treatment regimen, such as once-daily. See, e.g., Heise T and Meneghini L F, 20 ENDOCR. PRACT. p75-83 (2014).

In certain embodiments, the fusion protein of the present invention is administered in combination with an additional active ingredient, such as insulin or an insulin analog, an incretin-based therapy, or an oral diabetes medication, such as a sulfonylurea, metformin, thiazolidinedione such as pioglitazone or an α-glucosidase inhibitor such as acarbose.

The term “incretin-based therapy” includes any treatment which comprises administration of, or promotes, enables, enhances and/or simulates the effects of, a group of metabolic hormones known as incretins, which group includes GLP-1 and gastric inhibitory peptide (GIP). Incretin-based therapies which are currently available include DPP-4inhibitors and GLP-IR agonists.

A “DPP-4 inhibitor” is a compound that blocks the DPP-4 enzyme, which is responsible for the degradation of incretins. Currently available DPP-4 inhibitors include sitagliptin (Januvia®), and linagliptin (Tradjenta®).

A “GLP-1R agonist” is defined as a compound comprising the amino acid sequence of native human GLP-1 (SEQ ID NO:25), a GLP-1 analog, GLP-1 derivative or GLP-1 fusion protein, which maintains activity at the GLP-1 receptor. GLP-IR activity may be measured by methods known in the art, including using in vivo experiments and in vitro assays that measure GLP-1 receptor binding activity or receptor activation, e.g., assays employing pancreatic islet cells or insulinoma cells, as described in EP 619,322 and U.S. Pat. No. 5,120,712, respectively. A GLP-1 analog is a molecule having a modification including one or more amino acid substitutions, deletions, inversions, or additions when compared with the amino acid sequence of native human GLP-1 (SEQ ID NO:25). A GLP-1 derivative is a molecule having the amino acid sequence of native human GLP-1 (SEQ ID NO:25) or of a GLP-1 analog, but additionally having at least one chemical modification of one or more of its amino acid side groups, α-carbon atoms, terminal amino group, or terminal carboxylic acid group. A GLP-1 fusion protein is a heterologous protein comprising GLP-1, a GLP-1 analog or a GLP-1 derivative portion and a second polypeptide. Currently available GLP-IR agonists include exenatide (Byetta® and Bydureon®), liraglutide (Victoza®), albiglutide (Tanzeum®) and dulaglutide (Trulicity®), the structures of which are known in the art. See, e.g., U.S. Pat. No. 5,424,286 (exenatide); U.S. Pat. No. 6,268,343 (liraglutide); US 2014044717 (albiglutide); and U.S. Pat. No. 7,452,966 (dulaglutide).

In embodiments wherein a fusion protein of the present invention is provided in combination with an additional active ingredient, the fusion protein and additional active ingredient may be administered simultaneously, sequentially or in a single, combined formulation.

The fusion proteins of the present invention are effective in treating such diseases and conditions by administering to a patient in need thereof a therapeutically effective amount of a fusion protein of the present invention. As used herein, the phrase “therapeutically effective amount” refers to that amount of a fusion protein of the present invention sufficient to regulate blood glucose in a patient without causing unacceptable side effects. A therapeutically effective amount of the fusion protein administered to a subject will depend on the type and severity of the disease and on the characteristics of the subject, such as general health, age, sex, body weight, and tolerance to drugs. The skilled artisan will be able to determine appropriate dosages depending on these and other factors. In certain embodiments, a therapeutically effective amount of a fusion protein of the present invention when administered once weekly ranges from about 0.01 nmol/kg to about 100 nmol/kg. More preferably, a therapeutically effective amount of a fusion protein of the present invention when administered once weekly ranges from about 1 nmol/kg to about 50 nmol/kg. More preferably, a therapeutically effective amount of a fusion protein of the present invention when administered once weekly ranges from about 16 nmol/kg to about 25 nmol/kg. In certain embodiments, a therapeutically effective amount of a fusion protein of the present invention when administered once weekly ranges from about 1 mg to about 200 mg. More preferably, a therapeutically effective amount of a fusion protein of the present invention when administered once weekly ranges from about 25 mg to about 175 mg. More preferably, a therapeutically effective amount of a fusion protein of the present invention when administered once weekly ranges from about 100 mg to about 160 mg.

Persons of skill in the art will understand that when the fusion protein of the present invention is administered in combination with another active ingredient, such as a GLP-1R agonist, the dose may be adjusted so that the activity of the two treatments combined is sufficient to regulate blood glucose in a patient. Thus, the amount of fusion protein that must be administered to regulate blood glucose levels in such combinations may be less than would be required if the fusion protein were administered as a monotherapy. For example, when the fusion protein of the present invention is provided in combination with a GLP-1R agonist, the amount of fusion protein to be provided in a once weekly dose may be reduced by up to 50%, as compared to the amount of the same fusion protein for use as a monotherapy, such as the doses described in the preceding paragraph.

Preferably, administration of a therapeutically effective amount of the fusion protein of the present invention in some embodiments will provide effective glucose control while reducing the risk of hypoglycemia and/or weight gain as compared to existing treatments. The incidence of hypoglycemia caused by a diabetes therapy which agonizes the insulin receptor may be minimized by avoiding a rapid spike in the concentration of the therapeutic agent following administration. The fusion proteins of the present invention have an extended time action profile without a rapid spike in concentration following administration.

Moreover, in embodiments wherein the fusion proteins of the present invention are provided in combination with another active ingredient, in particular, a GLP-IR agonist, the hepatopreferential activity of the fusion proteins of the present invention may also reduce the risk of hypoglycemia while controlling glucose levels. Because the fusion protein of the present invention can readily access the liver through the fenestrated sinusoid endothelium, the molecule can control hepatic glucose output, with little, if any, activity at the periphery, while the GLP-IR agonist promotes glucose-dependent secretion of endogenous insulin from the pancreas that is readily capable of perfusing into the periphery to control glucose uptake in muscle and fat.

The fusion proteins of the present invention are administered parenterally, by nasal administration or pulmonary inhalation. Parenteral administration is preferred, and can include, for example, systemic administration, such as by intramuscular, intravenous, subcutaneous, or intraperitoneal injection.

The fusion proteins can be administered to the subject in a pharmaceutical composition, which comprises a fusion protein of the present invention and at least one pharmaceutically acceptable excipient. Such pharmaceutical compositions are typically, though not necessarily, parenteral in nature and may be prepared by any of a variety of techniques using conventional excipients for parenteral products, which are well known in the art. See, e.g., Remington: the Science and Practice of Pharmacy (D.B. Troy, Editor, 21^{st} Edition, Lippincott, Williams & Wilkins, 2006). As described above, the fusion protein of the present invention is a homodimer when expressed in mammalian cells. Thus, when used herein, the term “composition comprising a fusion protein” includes a composition, which contains a homodimer of a fusion protein. In certain embodiments, a pharmaceutical composition of the present invention includes a composition with the fusion protein of the present invention present in a concentration of at least 1 mg/mL, at least 2 mg/mL, at least 5 mg/mL, at least 10 mg/mL, at least 20 mg/mL, at least 25 mg/mL, at least 30 mg/mL, at least 35 mg/mL, at least 50 mg/mL, at least 55 mg/mL, at least 50 mg/mL, at least 65 mg/mL, at least 75 mg/mL, at least 100 mg/mL or greater. In preferred embodiments, the fusion protein is present in a concentration of 10-100 mg/mL. In more preferred embodiments, the fusion protein is present in a concentration of 15-75 mg/mL, and in most preferred embodiments, the fusion protein is present in a concentration of 20-65 mg/mL.

The term “excipient” means any substance added to the composition other than the fusion protein or any other additional active ingredient(s). Examples of such excipients that may be used in the compositions of the present invention include buffering agents, surfactants, isotonicity agents and preservatives.

In certain embodiments, the composition of the present invention includes one or more buffering agents to control the pH of the composition. A “buffering agent” is a substance which resists changes in pH by the action of its acid-base conjugate components. In certain embodiments, the composition of the present invention has a pH from about 5.5to about 8.0, preferably, between about 6.0 and about 7.4, more preferably between about 6.0 and 6.75. Buffering agents suitable for controlling the pH of the compositions of the present invention in the desired range include, but are not limited to agents such as phosphate, acetate, citrate, or acids thereof, arginine, TRIS, and histidine buffers, as well as combinations thereof. “TRIS” refers to 2-amino-2-hydroxymethyl-1,3,-propanediol, and to any pharmacologically acceptable salt thereof. The free base and the hydrochloride form (i.e., TRIS-HCl) are two common forms of TRIS. TRIS is also known in the art as trimethylol aminomethane, tromethamine, and tris (hydroxymethyl) aminomethane. Preferred buffering agents in the composition of the present invention are citrate, or citric acid, and phosphate.

In certain embodiments, the compositions of the present invention include one or more isotonicity agents to minimize pain upon injection due to cellular swelling or cellular rupture. An “isotonicity agent” is a compound that is physiologically tolerated and imparts a suitable tonicity to a formulation to prevent the net flow of water across cell membranes that are in contact with an administered pharmaceutical composition. Known isotonicity agents include glycerol, salts such as sodium chloride, and monosaccharides including, but not limited to, mannitol, dextrose and lactose. Preferred isotonicity agent(s) are mannitol and sodium chloride.

In certain embodiments, the compositions of the present invention include a surfactant. A “surfactant” is a substance that lowers the surface tension of a liquid. Examples of surfactants used in pharmaceutical compositions and which may be used in certain compositions of the present invention include polysorbate 20, polysorbate 80, polyethylene glycols (e.g., PEG 400, PEG 3000, TRITON X-100), polyethylene glycol alkyl ethers (e.g., BRIJ), polypropylene glycols, block copolymers (e.g., poloxamer, PLURONIC F68; poloxamer 407, PLURONIC F127; TETRONICS), sorbitan alkyl esters (e.g., SPAN), polyethoxylated castor oil (e.g., KOLLIPHOR, CREMOPHOR), and trehalose.

The pharmaceutical compositions of the present invention may also contain a preservative. The term “preservative” refers to a compound added to a pharmaceutical formulation to act as an anti-microbial agent in multi-use and/or titratable compositions. Among preservatives known in the art as being effective and acceptable in parenteral formulations are benzalkonium chloride, benzethonium, chlorohexidine, phenol, m-cresol, benzyl alcohol, methyl-or propyl-paraben, chlorobutanol, o-cresol, p-cresol, chlorocresol, phenylmercuric nitrate, thimerosal, benzoic acid, and various mixtures thereof. Phenolic preservative includes the compounds phenol, m-cresol, o-cresol, p-cresol, chlorocresol, methylparaben, and mixtures thereof. If a preservative is necessary, the preservative used in compositions of the present invention is preferably a phenolic preservative, preferably either m-cresol or phenol.

Certain phenolic preservatives, such as phenol and m-cresol, are known to bind to insulin and insulin hexamers and thereby stabilize a conformational change that increases either physical or chemical stability, or both. In compositions comprising other proteins, however, such preservatives may contribute to the formation of protein aggregates, or high molecular weight polymers (HMWP). See, e.g., Maa Y F and Hsu C C, Int J Pharm 140:155-168 (1996); Fransson J, et al., Pharm. Res., 14:606-612 (1997); Lam X M, et al., Pharm. Res., 14:725-729 (1997); Remmele R L Jr, et al., Pharm Res 15:200-208. (1998); Thirumangalathu R, et al., J Pharm Sci 95:1480-1497 (2006). Such protein aggregates in therapeutic formulations are undesirable due to their tendency to induce an immune response.

In certain preferred embodiments, the amino acid sequence of the fusion protein of the present invention is optimized to enhance the physical stability of the fusion protein in compositions which also contain a phenolic preservative. For example, the present inventors surprisingly discovered that the presence of an amino acid mutation at position 10 in the analog of the insulin A-chain (variable X₁ in the analog of the insulin A-chain recited in SEQ ID NO:2) reduces the accumulation of aggregates of the fusion protein in the presence of phenolic preservatives, as shown in the stability studies below.

The invention is further illustrated by the following examples, which are not to be construed as limiting.

### Expression and Purification of Fusion Proteins

Fusion proteins of the present invention are produced in a mammalian cell expression system using the CHO glutamine synthetase (GS) knockout (GSKO) cell line. The GS gene knockout enables tightened selection stringency by eliminating endogenous GS background activity which can allow survival of low-or non-productive cells under selection conditions. Genes coding for fusion proteins are sub-cloned into the glutamine synthetase (GS) containing expression plasmid. The cDNA sequence encoding the fusion proteins is fused in frame with the coding sequence of a signal peptide which enhances secretion of the fusion protein into the cell culture medium. The expression is driven by the cytomegalovirus (CMV) promoter. CHO GSKO cells are stably transfected using electroporation and the appropriate amount of recombinant expression plasmid.

Transfected cells undergo bulk selection in glutamine-free media. Transfected pools are plated at low density to allow for close-to-clonal outgrowth of stable expressing cells. The masterwells are screened for fusion protein expression and scaled-up in serum-free suspension cultures to be used for production.

Fusion proteins secreted into the media may be purified by Protein A affinity chromatography followed by size exclusion chromatography following standard chromatographic techniques. Briefly, fusion proteins from clarified media are captured by Mab Select Protein A (GE) that has been equilibrated with phosphate buffered saline pH 7.4. Following a wash step with phosphate buffered saline pH 7.4, bound fusion proteins are eluted with 10 mM citric acid pH 3.0. Fractions containing fusion protein are pooled and neutralized by adding 1/10 volume of 1M Tris pH 8.0. Soluble aggregates and multimers may be effectively removed by common techniques, including size exclusion, hydrophobic interaction or ion exchange chromatography. Fractions containing monomeric fusion protein (covalently linked homodimer), as determined by size exclusion chromatography, are pooled, sterile filtered, and stored.

Amino acid sequences of exemplary fusion proteins of the present invention are shown below:

### Example 1:

```
(SEQ ID NO: 12)
                10 20 30 40 50 60
FVNQHLCGSHLVEALELVCGERGFHYGGGGGGSGGGGGIVEQCCTSTCSLDQLENYCGGG
                70 80 90 100 110 120
GGQGGGGQGGGGQGGGGGECPPCPAPPVAGPSVFLFPPKPKDTLMISRTPEVTCVVVDVS
                130 140 150 160 170 180
HEDPEVQFNWYVDGVEVHNAKTKPREEQFNSTFRVVSVLTVVHQDWLNGKEYKCKVSNKG
                190 200 210 220 230 240
LPAPIEKTISKTKGQPREPQVYTLPPSREEMTKNQVSLTCLVKGFYPSDIAVEWESNGQP
                250 260 270 280 290
ENNYKTTPPMLDSDGSFFLYSKLTVDKSRWQQGNVFSCSVMHEALHNHYTQKSLSLSPG
```

### Example 2:

```
(SEQ ID NO: 16)
                10 20 30 40 50 60
FVNQHLCGSHLVEALYLVCGERGFFYTEETGGGGGGGIVEQCCTSICSLYQLENYCGGGG
                70 80 90 100 110 120
GSGGGGSGGGGSESKYGPPCPPCPAPEAAGGPSVFLFPPKPKDTLMISRTPEVTCVVVDV
                130 140 150 160 170 180
SQEDPEVQFNWYVDGVEVHNAKTKPREEQFNSTYRVVSVLTVLHQDWLNGKEYKCKVSNK
                190 200 210 220 230 240
GLPSSIEKTISKAKGQPREPQVYTLPPSQEEMTKNQVSLTCLVKGFYPSDIAVEWESNGQ
                250 260 270 280 290
PENNYKTTPPVLDSDGSFFLYSRLTVDKSRWQEGNVFSCSVMHEALHNHYTQKSLSLSLG
```

### Example 3:

```
(SEQ ID NO: 17)
                10 20 30 40 50 60
FVNQHLCGSHLVEALELVCGERGFHYGGGGGGSGGGGGIVEQCCTSICSLDQLENYCGGG
                70 80 90 100 110 120
GGQGGGGQGGGGQGGGGQGGPCPPCPAPEAAGGPSVFLFPPKPKDTLMISRTPEVTCVVV
                130 140 150 160 170 180
DVSQEDPEVQFNWYVDGVEVHNAKTKPREEQFNSTYRVVSVLTVLHQDWLNGKEYKCKVS
                190 200 210 220 230 240
NKGLPSSIEKTISKAKGQPREPQVYTLPPSQEEMTKNQVSLTCLVKGFYPSDIAVEWESN
                250 260 270 280 290 300
GQPENNYKTTPPVLDSDGSFFLYSRLTVDKSRWQEGNVFSCSVMHEALHNHYTQKSLSLS
LG
```

### Example 4:

```
(SEQ ID NO: 18)
                10 20 30 40 50 60
FVNQHLCGSHLVEALELVCGERGFHYGGGGGSGGGGGIVEQCCTSICSLDQLENYCGGGG
                70 80 90 100 110 120
GEGGGGEGGGGEGGGGECPPCPAPPVAGPSVFLFPPKPKDTLMISRTPEVTCVVVDVSHE
                130 140 150 160 170 180
DPEVQFNWYVDGVEVHNAKTKPREEQFNSTFRVVSVLTVVHQDWLNGKEYKCKVSNKGLP
                190 200 210 220 230 240
APIEKTISKTKGQPREPQVYTLPPSREEMTKNQVSLTCLVKGFYPSDIAVEWESNGQPEN
                250 260 270 280 290
NYKTTPPMLDSDGSFFLYSKLTVDKSRWQQGNVFSCSVMHEALHNHYTQKSLSLSPG
```

### Example 5:

```
(SEQ ID NO: 19)
                10 20 30 40 50 60
FVNQHLCGSHLVEALELVCGERGFHYGGGGGGSGGGGGIVEQCCTSTCSLDQLENYCGGG
                70 80 90 100 110 120
GGQGGGGQGGGGQGGGGQGGPCPPCPAPEAAGGPSVFLFPPKPKDTLMISRTPEVTCVVV
                130 140 150 160 170 180
DVSQEDPEVQFNWYVDGVEVHNAKTKPREEQFNSTYRVVSVLTVLHQDWLNGKEYKCKVS
                190 200 210 220 230 240
NKGLPSSIEKTISKAKGQPREPQVYTLPPSQEEMTKNQVSLTCLVKGFYPSDIAVEWESN
                250 260 270 280 290 300
GQPENNYKTTPPVLDSDGSFFLYSRLTVDKSRWQEGNVFSCSVMHEALHNHYTQKSLSLS
LG
```

### Example 6

```
(SEQ ID NO: 20)
                10 20 30 40 50 60
FVGQHLCGSHLVEALELVCGERGFHYGGGGGGSGGGGGIVEQCCTSICSLDQLENYCGGG
                70 80 90 100 110 120
GGQGGGGQGGGGQGGGGQGGPCPPCPAPEAAGGPSVFLFPPKPKDTLMISRTPEVTCVVV
                130 140 150 160 170 180
DVSQEDPEVQFNWYVDGVEVHNAKTKPREEQFNSTYRVVSVLTVLHQDWLNGKEYKCKVS
                190 200 210 220 230 240
NKGLPSSIEKTISKAKGQPREPQVYTLPPSQEEMTKNQVSLTCLVKGFYPSDIAVEWESN
                250 260 270 280 290 300
GQPENNYKTTPPVLDSDGSFFLYSRLTVDKSRWQEGNVFSCSVMHEALHNHYTQKSLSLS
LG
```

### Example 7

```
(SEQ ID NO: 21)
                10 20 30 40 50 60
AGGQHLCGSHLVEALELVCGERGFHYGGGGGSGGGGGIVEQCCTSICSLDQLENYCGGGG
                70 80 90 100 110 120
GQGGGGQGGGGQGGGGECPPCPAPPVAGPSVFLFPPKPKDILMISRTPEVTCVVVDVSHE
                130 140 150 160 170 180
DPEVQFNWYVDGVEVHNAKTKPREEQFNSTFRVVSVLTVVHQDWLNGKEYKCKVSNKGLP
                190 200 210 220 230 240
APIEKTISKTKGQPREPQVYTLPPSREEMTKNQVSLTCLVKGFYPSDIAVEWESNGQPEN
                250 260 270 280 290
NYKTTPPMLDSDGSFFLYSKLTVDKSRWQQGNVFSCSVMHEALHNHYTQKSLSLSPG
```

### Example 8

```
(SEQ ID NO: 22)
                  10 20 30 40 50 60
FVNQHLCGSHLVEALELVCGERGFHYTPKTGGSGGGGGIVEQCCTSTCSLDQLENYCGGG
                  70 80 90 100 110 120
GGQGGGGQGGGGQGGGGGECPPCPAPPVAGPSVFLFPPKPKDTLMISRTPEVTCVVVDVS
                  70 80 90 100 110 120
HEDPEVQFNWYVDGVEVHNAKTKPREEQFNSTFRVVSVLTVVHQDWLNGKEYKCKVSNKG
                  70 80 90 100 110 120
LPAPIEKTISKTKGQPREPQVYTLPPSREEMTKNQVSLTCLVKGFYPSDIAVEWESNGQP
                  70 80 90 100 110 120
ENNYKTTPPMLDSDGSFFLYSKLTVDKSRWQQGNVFSCSVMHEALHNHYTQKSLSLSPG
```

### Example 9

```
(SEQ ID NO: 23)
                  10 20 30 40 50 60
FVNQHLCGSHLVEALELVCGERGFFYTEETGGGGGGGIVEQCCTSICSLYQLENYCGGGG
                  70 80 90 100 110 120
GSGGGGSGGGGSGGGGSECPPCPAPPVAGPSVFLFPPKPKDTLMISRTPEVTCVVVDVSH
                    130 140 150 160 170 180
EDPEVQFNWYVDGVEVHNAKTKPREEQFNSTFRVVSVLIVVHQDWLNGKEYKCKVSNKGL
                    190 200 210 220 230 240
PAPIEKTISKTKGQPREPQVYTLPPSREEMTKNQVSLTCLVKGFYPSDIAVEWESNGQPE
                    250 260 270 280 290
NNYKTTPPMLDSDGSFFLYSKLTVDKSRWQQGNVFSCSVMHEALHNHYTQKSLSLSPG
```

### Example 10

```
(SEQ ID NO: 24)
                  10 20 30 40 50 60
FVGQHLCGSHLVEALELVCGERGFHYGGGGGGSGGGGGIVEQCCTSTCSLDQLENYCGGG
                  70 80 90 100 110 120
GGQGGGGQGGGGQGGGGGECPPCPAPPVAGPSVFLFPPKPKDILMISRTPEVTCVVVDVS
                  130 140 150 160 170 180
HEDPEVQFNWYVDGVEVHNAKTKPREEQFNSTFRVVSVLTVVHQDWLNGKEYKCKVSNKG
                  190 200 210 220 230 240
LPAPIEKTISKTKGQPREPQVYTLPPSREEMTKNQVSLICLVKGFYPSDIAVEWESNGQP
                  250 260 270 280 290
ENNYKTITPMLDSDGSFFLYSKLTVDKSRWQQGNVFSCSVMHEALHNHYTQKSLSLSPG
```

### In Vitro Activity

Test-lots of Examples 1-3, 5 and 9 are prepared in phosphate buffered saline (PBS, pH 7.4) or 10 mM citrate buffer (pH 6.5) and stored at 4° C. Biosynthetic human insulin (Eli Lilly and Company) is prepared in 0.01 N HCl and stored as frozen aliquots, or prepared in a formulated mixture containing m-cresol, zinc, sodium chloride, and TRIS buffer (pH 7.3) at 100 units/mL and stored at 4° C.

Affinities of fusion proteins are determined in receptor binding assays performed on P1 membranes prepared from stably-transfected 293EBNA cells (293HEK human embryonic kidney cells expressing EBNA-1) over-expressing either human insulin receptor isoform A (hIR-A) or human insulin receptor isoform B (hIR-B) containing a C9 epitope tag at the C-terminus. Binding affinities are determined from a competitive radio-ligand

binding assay performed at steady-state using human recombinant (3-[¹²⁵I]-iodotyrosyl-A¹⁴)-insulin. Values for test samples are calculated as percent relative to the activity of unlabeled human insulin. IC₅₀ values are determined from 4-parameter logistic non-linear regression analysis (XLFit version 4.0, IDBS). If necessary, curve top or bottom parameters are set to 100 or 0, respectively.

The affinity constant (Ki) is calculated from the IC₅₀ value based upon the equation Kᵢ=IC₅₀/(1+D/K_{d}) where D equals the concentration of radio-ligand used in the experiment and K_{d} equals the equilibrium binding affinity constant of the radio-ligand for its cognate receptor determined from saturation binding analysis (hIR-A=0.205 nM; hIR-B=0.251 nM). Reported values for Kᵢ are shown as geometric mean±the standard error of the mean (SEM), with the number of replicate determinations indicated by “n” (Table 1).

The exemplified fusion proteins have affinity at both hIR-A and hIR-B. Compared to human insulin, the exemplified fusion proteins show reduced binding affinity to hIR-A and hIR-B (Table 1).

TABLE 1

| | Receptor Binding Affinity, | |
|---|---|---|
| | Ki, nM (SEM, n) | |
| Sample | hIR-A | hIR-B |
| Example 1 | 24.9 (4.3, n = 10) | 26.2 (4.3, n = 10) |
| Example 2 | 1.61 (0.06, n = 3) | 4.60 (0.86, n = 3) |
| Example 3 | 10.1 (1.5, n = 4) | 14.5 (2.3, n = 4) |
| Example 5 | 35.6 (10.4, n = 4) | 24.6 (3.4, n = 4) |
| Example 9 | 3.74 (1.20, n = 2) | 4.71 (1.78, n = 2) |
| Human insulin | 0.166 (0.008, n = 10) | 0.202 (0.007, n = 10) |

Ki values are geometric means.
SEM is standard error of the mean.
n is the number of observations.

### In Vivo Studies

### Studies in Streptozotocin (STZ)-Treated Rat Diabetes Model

Effects of Fusion Proteins are investigated in STZ-treated rat diabetes model. Male Sprague-Dawley rats, 400-425 gram body weight, are anesthetized with isoflurane and given a single injection of Zanosar® (STZ item #89256, Teva Parenteral Medicines, 40 mg/kg IV). The rats are used in studies 3 days after injection of Zanosar®; only animals with non-fasted blood glucose between 400-550 mg/dL are used in these studies.

The rats are distributed into groups to provide comparable variance in blood glucose and body weight and then randomized. Blood glucose is measured using Accucheck Aviva glucometer (Roche). STZ-treated rats are given a single subcutaneous (SC) injection of 30 nmol/kg dose.

Blood samples for glucose measurements are collected by tail bleed. Animals have free access to food and water throughout the experiment. Blood glucose data are provided in FIG. 1. Data shown in FIG. 1 are mean±SEM (n=6 for Examples 1 and 8; n=3 for remaining examples). Blood glucose data for time points during the initial feeding period (between 0 and 24 hours) are collected, but are not included in FIG. 1 for ease of visual representation. As shown in FIG. 1, Examples 1-10 each provide glucose lowering for a prolonged period of time.

Pharmacokinetic properties of Examples 1, 3-6 and 9-10 are also characterized following subcutaneous (SC) dosing in STZ-treated rats as described above. Data are generated using an insulin receptor ELISA assay that requires the presence of insulin that is capable of binding the insulin receptor. The insulin receptor ELISA utilizes the human insulin receptor (R&D Systems #1544-IR/CF aa28-944) as capture. The human insulin receptor is attached to an Immunlon 4 HBX plate by mouse anti-6x HisTag antibody (Novagen 70796). Fusion protein standard curve and samples are diluted in 100% rat K3 EDTA plasma and detected by mouse anti-human IgG Fc horseradish peroxidase (SouthernBiotech 9040-05). Concentrations of Examples 3-6 and 9-10 at 336 hour time point are all between 22.1±9.8 mg/mL and 1498±690 mg/mL. Table 3 shows concentrations of Example 1 over time, and Table 4 shows pharmacokinetic parameters following non-compartmental analysis of the data for Example 1. The data support an extended duration of bioavailability for fusion proteins of the present invention.

TABLE 3

| Time | |
|---|---|
| (Hours) | Concentration (mg/mL) |
| 1 | 335 ± 104 |
| 6 | 3559 ± 447 |
| 12 | 5991 ± 1578 |
| 24 | 10614 ± 1334 |
| 48 | 12629 ± 1811 |
| 96 | 8766 ± 2028 |
| 168 | 5017 ± 253 |
| 240 | 3682 ± 509 |
| 336 | 2014 ± 134 |

Data represent mean and standard deviation of n = 3.

TABLE 4

| PK Parameter | Result |
|---|---|
| AUC_{0-∞} (μg*hr/mL) | 1066 ± 363 |
| Cₘₐₓ (μg/mL) | 6.81 ± 1.62 |
| Tₘₐₓ (hr) | 40 ± 14 |
| CL or CL/F | 2.15 ± 0.91 |
| (mL/hr/kg) | |
| t_{1/2} (hr) | 82 ± 12 |
| % F | 147 |

Data represent mean and standard deviation of n = 3.
Abbreviations: AUC_{0-∞}—area under the curve from 0 to infinity, Cₘₐₓ—maximal concentration, Tₘₐₓ—time at maximum concentration, CL—clearance, F—bioavailability, t½—half-life.

### Euglycemic Clamp Study in Normal Rats

A euglycemic clamp study in male Sprague-Dawley rats is performed to understand overall in vivo activity of Example 1 on glucose utilization and to determine activity of Example 1 in liver versus peripheral tissues. A bolus/continuous infusion of 3-3H-glucose is initiated in chronically catheterized, overnight-fasted rats to determine endogenous glucose production (EGP) under basal conditions. A bolus/continuous intravenous infusion of test article—7 nmol/kg [bolus] and Inmol/kg/hr [continuous rate]—or insulin lispro comparator—[no bolus] and 0.75 mU/kg/min [continuous rate]—is then administered and a variable intravenous infusion of 20% glucose is initiated and periodically adjusted to maintain blood glucose concentration at 100-110 mg/dL. Bolus/infusion rates are selected to achieve comparable glucose infusion rates (GIR) and comparable suppression of EGP under euglycemic clamp conditions in each group. Somatostatin is administered to inhibit endogenous insulin secretion. Arterial blood samples are obtained during the experiment to monitor hematocrit, plasma insulin, and free fatty acids, C-peptide, and basal and clamp EGP. At the end of the experiment, 2-[1-¹⁴C]-deoxyglucose is administered to measure tissue glucose uptake under steady state glucose concentrations. Under these matched conditions, peripheral activity of the test article and comparators is analyzed as glucose uptake in the soleus muscle and suppression of plasma free fatty acids (FFA). All data are analyzed using a one-way Analysis of Variance with Dunnett's post-hoc test using the insulin lispro group as the control comparator.

Bolus and infusion doses of Example 1 result in steady plasma concentration of 274±57 nM over the course of the clamp experiment. As shown in Table 5, both groups of animals achieve comparable GIR during the clamp phase of the experiment. Average blood glucose in the Example 1 group is slightly higher than the insulin lispro group. Both groups have similar basal and clamped EGP rates (Table 5). Furthermore, the percent change from basal EGP in the Example 1 group is comparable to the insulin lispro control group (Table 5). To assess peripheral activity under equivalently clamped conditions, suppression of plasma FFA and muscle glucose uptake are measured. Whereas insulin lispro resulted in a reduction in plasma FFA levels over the course of the clamp experiment, Example 1 did not. (Table 5). In addition, infusion of Example 1 results in a 33% decrease in glucose uptake in the soleus muscle compared to insulin lispro. (Table 5). Collectively, these data indicate Example 1 displays reduced peripheral activity compared to insulin lispro.

TABLE 5

| | | Insulin lispro | Example 1 |
|---|---|---|---|
| Blood Glucose (mg/dL) | | 106.3 ± 2.2 | 111.8 ± 1.2 * |
| GIR (mg/kg/min) | | 4.71 ± 0.46 | 4.72 ± 0.13 |
| EGP Rate (mg/kg/min) | Basal | 5.062 ± 0.141 | 5.004 ± 0.093 |
| | Clamp | 3.607 ± 0.241 | 3.509 ± 0.160 |

| | | |
|---|---|---|
| % Change from Basal EGP | −29.3 ± 3.5 | −30.1 ± 2.5 |
| % Suppression plasma FFA from Basal | −14.5 ± 4.1 | 9.9 ± 2.4 * |
| Rg (μmo1/100 mg/min) | 11.34 ± 1.73 | 7.66 ± 1.38 |

Values are displayed as mean ± SEM of 13 animals for insulin lispro and 17 animals for Example 1.
Rg = glucose metabolic index.
Statistical analysis was completed by one-way ANOVA followed by Dunnett's post-hoc test.
* = Significantly different from insulin lispro (p < 0.05).

### In vivo Efficacy in Cynomolgus Monkeys

The pharmacokinetic (PK) parameters of Example 1 are evaluated following a single intravenous dose of 1.5 nmol/kg and a single subcutaneous dose of 3 nmol/kg in cynomolgus monkeys. Plasma samples for the PK analysis are collected from three animals per group/route, over the course of 3 weeks. Two assays are utilized for the PK analysis: insulin receptor ELISA and total IgG Fc ELISA. The insulin receptor ELISA utilizes the human insulin receptor (R&D Systems1544-IR/CF) as capture. The human insulin receptor is attached to an Immunlon 4 HBX plate by mouse anti-HisTag antibody (Novagen 70796). Example 1 standard curve and samples are diluted in 100% cynomolgus monkey plasma (anticoagulant was K3 EDTA) and detected by mouse anti-human IgG Fc horseradish peroxidase (SouthernBiotech 9040-05). The total IgG ELISA utilizes the anti-human IgG₂ (Abcam ab 1933) as the capture reagent. Example 1 is diluted in 100% cynomolgus monkey plasma and the detection antibody is the same as in the insulin receptor ELISA. Results from both assays are shown in Table 6, and the associated PK parameters are shown in Table 7. Example 1 shows complete bioavailability in monkeys, and the active insulin assay and the total Fc assay give similar results.

TABLE 6
PK of Example 1 in Normal Cynomolgus Monkeys.

| | Concentration ± SD (ng/mL) | | | |
|---|---|---|---|---|
| | Insulin Receptor ELISA | | Total Fc ELISA | |
| | IV | SC | IV | SC |
| Time (hours) | 1.5 nmol/kg | 3.0 nmol/kg | 1.5 nmol/kg | 3.0 nmol/kg |
| 0.083 | 1213 ± 212 | NA | 2190 ± 1369 | NA |
| 1 | 1172 ± 182 | <43.75 ± NC | 1708 ± 1219 | <43.75 ± NC |
| 3 | 863 ± 114 | 87 ± 25 | 798 ± NC | 92 ± 22 |
| 6 | 726 ± 119 | 272 ± 31 | 688 ± 96 | 209 ± 116 |
| 12 | 576 ± 88 | 579 ± 78 | 457 ± 178 | 409 ± 145 |
| 24 | 422 ± 67 | 788 ± 69 | 332 ± 95 | 685 ± 359 |
| 48 | 299 ± 39 | 805 ± 55 | 260 ± 19 | 802 ± 64 |
| 72 | 219 ± 42 | 651 ± 83 | 199 ± 34 | 770 ± 83 |
| 96 | 173 ± 26 | 544 ± 89 | 159 ± 25 | 703 ± 390 |
| 120 | 146 ± 28 | 452 ± 55 | 152 ± 27 | 574 ± 160 |
| 168 | 83 ± 13 | 289 ± 54 | 126 ± NC | 395 ± 70 |
| 216 | 48 ± NC | 183 ± 44 | 99 ± NC | 208 ± 123 |
| 240 | <43.75 ± NC | 145 ± 25 | 80 ± NC | 242 ± NC |
| 336 | <43.75 ± NC | 63 ± NC | 47 ± NC | NC |
| 504 | <43.75 ± NC | <43.75 ± NC | <43.75 ± NC | <43.75 ± NC |

Data represent mean and standard deviation from n = 3.
Abbreviations: IV = intravenous; SD = standard deviation; NC = not calculated

TABLE 7
PK Parameters derived from Non-compartmental Analysis of Data in
Table 6.

| | Insulin | | Total | |
|---|---|---|---|---|
| | Receptor ELISA | | Fc ELISA | |
| | IV | SC | IV | SC |
| Dose (nmol/kg) | 1.5 | 3.0 | 1.5 | 3.0 |
| AUC_{0-inf} (μg*hr/mL) | 51.1 ± 6.4 | 127 ± 7 | 62.7 ± 5.8 | 171 ± 17 |
| Cₘₐₓ (μg/mL) | 1.22 ± 0.22 | 0.82 ± 0.06 | 2.24 ± 1.38 | 0.90 ± 0.21 |
| Tₘₐₓ (hr) | 0 | 48 ± 24 | 0 | 64 ± 28 |
| CL or CL/F (mL/hr/kg) | 1.99 ± 0.24 | 1.59 ± 0.09 | 1.61 ± 0.15 | 1.19 ± 0.11 |
| T_{1/2} (hr) | 61 ± 9 | 70 ± 2 | 127 ± 18 | 148 ± 53 |
| Vss (mL/kg) | 164 ± 31 | NA | 256 ± 47 | NA |
| % F | NA | 125 | NA | 136 |

Data represent mean and standard deviation from n = 3.
Abbreviations: AUC_{0-inf}—area under the curve from 0 to infinity, Cₘₐₓ—maximal concentration (for IV administration Cₘₐₓ is extrapolated concentration at time 0), Tₘₐₓ—time at maximum concentration, CL—clearance, F—bioavailability, t½—half-life, Vₛₛ—steady-state volume of distribution, NA—not applicable.

### Stability

### Non-preserved 65 mg/mL Formulation of Example 1

Example 1 is formulated at 65 mg/mL in 10 mM citrate, 46.4 mg/mL mannitol, 0.02% polysorbate 80, pH 6.5 and stored at 30° C. Samples are analyzed for percent high molecular weight by size exclusion chromatography (SEC) at 0, 2, and 4 weeks by injecting 1 μL of the 65 mg/ml sample. Analytical SEC is performed on an Agilent 1100 system equipped with a TSKgel SuperSW3000 (Tosoh Bioscience) column and 50 mM sodium phosphate, 300 mM NaCl, pH 7.0 mobile phase flowing at 0.4 mL/min for 15 minutes. Peaks are detected at an absorbance of 280 nm and chromatograms are analyzed using ChemStation software. Percent high molecular weight at time zero is 1.13%, at time two weeks is 1.68%, and at time four weeks is 1.74%. These results support stability of Example 1 at 65 mg/mL concentration with minimal growth of soluble aggregate after 4 weeks at 30 C.

### Preserved and Non-Preserved 1 mg/mL Formulations of Examples 1 and 3

Examples 1 and 3 are formulated at 1 mg/mL in 10 mM citrate, pH 6.5 in the presence or absence of 30 mM m-Cresol and stored at 30° C. Samples are analyzed for percent high molecular weight by size exclusion chromatography (SEC) at time zero and 2weeks by injecting 10 μL of the 1 mg/mL sample. Analytical SEC is performed on an Agilent 1100 system equipped with a TSKgel G3000SWxl (Tosoh Bioscience) column and PBS+350 mM NaCl, pH 7.4 mobile phase flowing at 0.5 mL/min for 35 minutes or 45 minutes for samples in the absence or presence of m-cresol, respectively. Peaks are detected at an absorbance of 214 nm and chromatograms are analyzed using ChemStation software. For Example 3, without and with m-cresol, percent high molecular weight at time zero is 0.2% and 3.06%, respectively. Percent high molecular weight at 2 weeks without and with m-cresol was 0.2% and 1.73%, respectively. These results show an immediate increase in soluble aggregate after addition of m-cresol for Example 3 at time zero. For Example 1, in the absence and presence of m-cresol, percent high molecular weight at time zero was 0.16% and 0.15%, respectively. Percent high molecular weight at 2 weeks in the absence and presence of m-cresol was 0.18% and 0.31%, respectively. These results demonstrate stability, in the presence of preservative, of Example 1, which includes modification of the amino acid at position 10 in the analog of the insulin A-chain (variable X₁ in SEQ ID NO:2, Z₃ in the first fusion protein described above) to a T residue, relative to Example 3, which includes the native I residue at that position, but which otherwise comprises the same insulin receptor agonist amino acid sequence as Example 1.

### Sequences

Analog of insulin B-chain
SEQ ID NO: 1
X₁X₂X₃QHLCGSHLVEALX₄LVCGERGFX₅YX₆X₇X₈X₉

wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X₉ is G, T, S, E, K, A or is absent, provided that the insulin B-chain analog includes at least one modification from the amino acid sequence of the B-chain of a molecule of human insulin at X₄, X₅, X₆, X₇, X₈, or X₉

Analog of insulin A-chain
SEQ ID NO: 2
GIVEQCCTSX₁CSLX₂QLENYCX₃X₄

X₁ is T or I; X₂ is D, Y, Q or E; X₃ is G, N, S or A; and X₄ is any naturally occurring amino acid, or is absent, provided that if X₃ is N, then X₄ must be an amino acid other than Gor N

### SEQ ID NO:3—First Peptide Linker

X₁GX GGGG, wherein X₁ is G or is absent; and X₂ is G, S or is absent

Insulin receptor agonist
SEQ ID NO: 4
X₁X₂X₃QHLCGSHLVEALX₄LVCGERGFX₅YX₆X₇X₈X₉X₁₀GX₁₁GGGGGIVE
QCCTSX₁₂CSLX₁₃QLENYCX₁₄X₁₅

wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X₉ is G, T, S, E, K, A or is absent; X₁₀ is G or is absent; X₁₁ is G, S or is absent; X₁₂ is T or I; X₁₃ is D, Y, Q or E; X₁₄ is G, N, S or A; and X₁₅ is any naturally occurring amino acid, or is absent, provided that at least one of X₄, X₅, X₆, X₇, X₈, or X₉ must be a different amino acid than that found, respectively, at position B₁₆, B₂₅, B₂₇, B₂₈, B₂₉ or B₃₀ of the B-chain of a molecule of human insulin, and further provided that if X₁₄ is N, then X₁₅ must be an amino acid other than G or N

Insulin receptor agonist
SEQ ID NO: 5
FVNQHLCGSHLVEALELVCGERGFHYGGGGGGSGGGGGIVEQCCTSTCSL
DQLENYCG

Second peptide linker
SEQ ID NO: 6
GGGGX₁GGGGX₂GGGGX₃GGGGX₄X₅X₆

wherein X₁ is Q or E; X₂ is Q or E; X₃ is Q or E; X₄ is G, E, Q or is absent; X₅ is G or absent; and X₅ is G or is absent

Second peptide linker
SEQ ID NO: 7
GGGGQGGGGQGGGGQGGGGG

Human IgG Fc region
SEQ ID NO: 8
CPPCPAPELLGGPSVX₁LX₂PPKPKDTLMISRTPEVTCX₃VX₄DVSHEDPE
VKFNWYVDGVEVEINAKTKPREEQYX₅STYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGX₆

wherein X₁ is F, Q or E; X₂ is F, Q or E; X₃ is V or T; X₄ is V or T; X₅ is N, D or Q; and X₆ is K or is absent

Human IgG Fc region
SEQ ID NO: 9
PCPPCPAPEAAGGPSVX₁LX₂PPKPKDTLMISRTPEVTCX₃VX₄DVSQEDP
EVQFNWYVDGVEVHNAKTKPREEQFX₅STYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSX₆LTVDKSRWQE
GNVFSCSVMHEALHNHYTQKSLSLSLGX₇

wherein X₁ is F, Q or E; X₂ F, Q or E; X₃ is V or T; X₄ is V or T; X₅ is N, D or Q; X₆ is R, or K; X₇ is K or is absent

Human IgG Fc region
SEQ ID NO: 10
ECPPCPAPPVAGPSVX₁LX₂PPKPKDTLMISRTPEVTCX₃VX₄DVSHEDPE
VQFNWYVDGVEVHNAKTKPREEQFX₅STFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGX₆

wherein X₁ is F, Q or E; X₂ is F, Q or E; X₃ is V or T; X₄ is V or T; X₅ is N, D or Q; and X₆ is K or absent

X₁X₂X₃QHLCGSHLVEALX₄LVCGERGFX₅YX₆X₇X₈X₉X₁₀GX₁₁GGGGGIVEQ
CCTSX₁₂CSLX₁₃QLENYCX₁₄X₁₅GGGGX₁₆GGGGX₁₇GGGGX₁₈GGGGX₁₉X₂₀
X₂₁X₂₂CPPCPAPX₂₃X₂₄AGX₂₅PSVFLFPPKPKDTLMISRTPEVTCVVVDV
SX₂₆EDPEVQFNWYVDGVEVHNAKTKPREEQFNSTX₂₇RVVSVLTVX₂₈HQD
WLNGKEYKCKVSNKGLPX₂₉X₃₀IEKTISKX₃₁KGQPREPQVYTLPPSX₃₂EE
MTKNQVSLTCLVKGFYPSDIAVEWESNGQPENNYKTITPX₃₃LDSDGSFFL
YSX₃₄LTVDKSRWQX₃₅GNVFSCSVMHEALHNHYTQKSLSLSX₃₆G

wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X, is G, T, S, E, K, A or is absent, provided that at least one of X₄, X₅, X₆, X₇, X₈, or X₉ is an amino acid other than that which is present, respectively, at position B₁₆, B₂₅, B₂₇, B₂₈, B₂₉ or B₃₀ of a human insulin B-chain; X₁₀ is G or is absent; X₁₁ is G, S or is absent; X₁₂ is T or I; X₁₃ is D, Y, Q or E; X₁₄ is G, N, S or A; X₁₅ is any naturally occurring amino acid, or is absent, provided that if X₁₄ is N, then X₁₅ must be an amino acid other than G or N; X₁₆ is Q or E; X₁₇ is Q or E; X₁₈ is Q or E; X₁₉ is G, E, Q or is absent; X₂₀ is G or absent; X₂₁ is G or is absent; X₂₂ is E or P; X₂₃ is E or P; X₂₄ is A or V; X₂₅ is G or is absent; X₂₆ is Q or H; X₂₇ is Y or F; X₂₈ is L or V; X₂₉ is S or A; X₃₀ is S or P; X₃₁ is A or T; X₃₂ is Q or R; X₃₃ is V or M; X₃₄ is R or K; X₃₅ is E or Q; and X₃₆ is L or P

```
Fusion protein
SEQ ID NO: 12
                  10 20 30 40 50 60
FVNQHLCGSHLVEALELVCGERGFHYGGGGGGSGGGGGIVEQCCTSTCSLDQLENYCGGG
                  70 80 90 100 110 120
GGQGGGGQGGGGQGGGGGECPPCPAPPVAGPSVFLFPPKPKDTLMISRTPEVTCVVVDVS
                  130 140 150 160 170 180
HEDPEVQFNWYVDGVEVHNAKTKPREEQFNSTFRVVSVLTVVHQDWLNGKEYKCKVSNKG
                  190 200 210 220 230 240
LPAPIEKTISKTKGQPREPQVYTLPPSREEMTKNQVSLTCLVKGFYPSDIAVEWESNGQP
                  250 260 270 280 290
ENNYKTTPPMLDSDGSFFLYSKLTVDKSRWQQGNVFSCSVMHEALHNHYTQKSLSLSPG
```

Human insulin A-chain
SEQ ID NO: 13
GIVEQCCTSICSLYQLENYCN

Human insulin B-chain
SEQ ID NO: 14
FVNQHLCGSHLVEALYLVCGERGFFYTPKT

First peptide linker
SEQ ID NO: 15
GGSGGGG

```
Fusion protein
SEQ ID NO: 16
                  10 20 30 40 50 60
FVNQHLCGSHLVEALYLVCGERGFFYTEETGGGGGGGIVEQCCTSICSLYQLENYCGGGG
                  70 80 90 100 110 120
GSGGGGSGGGGSESKYGPPCPPCPAPEAAGGPSVFLFPPKPKDTLMISRTPEVTCVVVDV
                  130 140 150 160 170 180
SQEDPEVQFNWYVDGVEVHNAKTKPREEQFNSTYRVVSVLTVLHQDWLNGKEYKCKVSNK
                  190 200 210 220 230 240
GLPSSIEKTISKAKGQPREPQVYTLPPSQEEMTKNQVSLTCLVKGFYPSDIAVEWESNGQ
                  250 260 270 280 290
PENNYKTTPPVLDSDGSFFLYSRLTVDKSRWQEGNVFSCSVMHEALHNHYTQKSLSLSLG
```

```
Fusion protein
SEQ ID NO: 17
                  10 20 30 40 50 60
FVNQHLCGSHLVEALELVCGERGFHYGGGGGGSGGGGGIVEQCCTSICSLDQLENYCGGG
                  70 80 90 100 110 120
GGQGGGGQGGGGQGGGGQGGPCPPCPAPEAAGGPSVFLFPPKPKDTLMISRTPEVTCVVV
                  130 140 150 160 170 180
DVSQEDPEVQFNWYVDGVEVHNAKTKPREEQFNSTYRVVSVLTVLHQDWLNGKEYKCKVS
                  190 200 210 220 230 240
NKGLPSSIEKTISKAKGQPREPQVYTLPPSQEEMTKNQVSLTCLVKGFYPSDIAVEWESN
                  250 260 270 280 290 300
GQPENNYKTTPPVLDSDGSFFLYSRLTVDKSRWQEGNVFSCSVMHEALHNHYTQKSLSLS
LG
```

```
Fusion protein
SEQ ID NO: 12
                  10 20 30 40 50 60
FVNQHLCGSHLVEALELVCGERGFHYGGGGGSGGGGGIVEQCCTSTCSLDQLENYCGGGG
                  70 80 90 100 110 120
GEGGGGEGGGGEGGGGECPPCPAPPVAGPSVFLFPPKPKDTLMISRTPEVTCVVVDVSHE
                  130 140 150 160 170 180
DPEVQFNWYVDGVEVHNAKTKPREEQFNSTFRVVSVLTVVHQDWLNGKEYKCKVSNKGLP
                  190 200 210 220 230 240
APIEKTISKTKGQPREPQVYTLPPSREEMTKNQVSLTCLVKGFYPSDIAVEWESNGQPEN
                  250 260 270 280 290
NYKTTPPMLDSDGSFFLYSKLTVDKSRWQQGNVFSCSVMHEALHNHYTQKSLSLSPG
```

```
Fusion protein
SEQ ID NO: 19
                  10 20 30 40 50 60
FVNQHLCGSHLVEALELVCGERGFHYGGGGGGSGGGGGIVEQCCTSTCSLDQLENYCGGG
                  70 80 90 100 110 120
GGQGGGGQGGGGQGGGGQGGPCPPCPAPEAAGGPSVFLFPPKPKDTLMISRTPEVTCVVV
                  130 140 150 160 170 180
DVSQEDPEVQFNWYVDGVEVHNAKTKPREEQFNSTYRVVSVLTVLHQDWLNGKEYKCKVS
                  190 200 210 220 230 240
NKGLPSSIEKTISKAKGQPREPQVYTLPPSQEEMTKNQVSLTCLVKGFYPSDIAVEWESN
                  250 260 270 280 290 300
GQPENNYKTTPPVLDSDGSFFLYSRLTVDKSRWQEGNVFSCSVMHEALHNHYTQKSLSLS
LG
```

```
Fusion protein
SEQ ID NO: 20
                  10 20 30 40 50 60
FVGQHLCGSHLVEALELVCGERGFHYGGGGGGSGGGGGIVEQCCTSICSLDQLENYCGGG
                  70 80 90 100 110 120
GGQGGGGQGGGGQGGGGQGGPCPPCPAPEAAGGPSVFLFPPKPKDTLMISRTPEVTCVVV
                  130 140 150 160 170 180
DVSQEDPEVQFNWYVDGVEVHNAKTKPREEQFNSTYRVVSVLTVLHQDWLNGKEYKCKVS
                  190 200 210 220 230 240
NKGLPSSIEKTISKAKGQPREPQVYTLPPSQEEMTKNQVSLTCLVKGFYPSDIAVEWESN
                  250 260 270 280 290 300
GQPENNYKTTPPVLDSDGSFFLYSRLTVDKSRWQEGNVFSCSVMHEALHNHYTQKSLSLS
LG
```

```
Fusion protein
SEQ ID NO: 21
                  10 20 30 40 50 60
AGGQHLCGSHLVEALELVCGERGFHYGGGGGSGGGGGIVEQCCTSICSLDQLENYCGGGG
                  70 80 90 100 110 120
GQGGGGQGGGGQGGGGECPPCPAPPVAGPSVFLFPPKPKDTLMISRTPEVTCVVVDVSHE
                    130 140 150 160 170 180
DPEVQFNWYVDGVEVHNAKTKPREEQFNSTFRVVSVLTVVHQDWLNGKEYKCKVSNKGLP
                  190 200 210 220 230 240
APIEKTISKTKGQPREPQVYTLPPSREEMTKNQVSLTCLVKGFYPSDIAVEWESNGQPEN
                  250 260 270 280 290
NYKTTPPMLDSDGSFFLYSKLTVDKSRWQQGNVFSCSVMHEALHNHYTQKSLSLSPG
```

```
Fusion protein
SEQ ID NO: 22
                  10 20 30 40 50 60
FVNQHLCGSHLVEALELVCGERGFHYTPKTGGSGGGGGIVEQCCTSTCSLDQLENYCGGG
                  70 80 90 100 110 120
GGQGGGGQGGGGQGGGGGECPPCPAPPVAGPSVFLFPPKPKDTLMISRTPEVTCVVVDVS
                      130 140 150 160 170 180
HEDPEVQFNWYVDGVEVHNAKTKPREEQFNSTFRVVSVLTVVHQDWLNGKEYKCKVSNKG
                  190 200 210 220 230 240
LPAPIEKTISKTKGQPREPQVYTLPPSREEMTKNQVSLTCLVKGFYPSDIAVEWESNGQP
                  250 260 270 280 290
ENNYKTTPPMLDSDGSFFLYSKLTVDKSRWQQGNVFSCSVMHEALHNHYTQKSLSLSPG
```

```
Fusion protein
SEQ ID NO: 23
                  10 20 30 40 50 60
FVNQHLCGSHLVEALELVCGERGFFYTEETGGGGGGGIVEQCCTSICSLYQLENYCGGGG
                  70 80 90 100 110 120
GSGGGGSGGGGSGGGGSECPPCPAPPVAGPSVFLFPPKPKDTLMISRTPEVTCVVVDVSH
                    130 140 150 160 170 180
EDPEVQFNWYVDGVEVHNAKTKPREEQFNSTFRVVSVLTVVHQDWLNGKEYKCKVSNKGL
                    190 200 210 220 230 240
PAPIEKTISKTKGQPREPQVYTLPPSREEMTKNQVSLTCLVKGFYPSDIAVEWESNGQPE
                    250 260 270 280 290
NNYKTTPPMLDSDGSFFLYSKLTVDKSRWQQGNVFSCSVMHEALHNHYTQKSLSLSPG
```

```
Fusion protein
SEQ ID NO: 24
                  10 20 30 40 50 60
FVGQHLCGSHLVEALELVCGERGFHYGGGGGGSGGGGGIVEQCCTSTCSLDQLENYCGGG
                  70 80 90 100 110 120
GGQGGGGQGGGGQGGGGGECPPCPAPPVAGPSVFLFPPKPKDTLMISRTPEVTCVVVDVS
                    130 140 150 160 170 180
HEDPEVQFNWYVDGVEVHNAKTKPREEQFNSTFRVVSVLTVVHQDWLNGKEYKCKVSNKG
                    190 200 210 220 230 240
LPAPIEKTISKTKGQPREPQVYTLPPSREEMTKNQVSLTCLVKGFYPSDIAVEWESNGQP
                  250 260 270 280 290
ENNYKTTPPMLDSDGSFFLYSKLTVDKSRWQQGNVFSCSVMHEALHNHYTQKSLSLSPG
```

GLP-1
SEQ ID NO: 25
HAEGTFTSDVSSYLEGQAAKEFIAWLVKGRG

Second peptide linker
SEQ ID NO: 26
[GGGGX]ₙ, wherein X is Q, E or S; and wherein
n is 2-5

N-terminal end of wild-type IgG2 Fc region
SEQ ID NO: 27
ERKCCV

N-terminal end of wild-type IgG4 Fc region
SEQ ID NO: 28
ESKYGP

N-terminal end of wild-type IgG1 Fc region
SEQ ID NO: 29
EPKSCDKTHT

We claim:

1. A fusion protein comprising:
a) an insulin receptor agonist having the general formula Z₁-Z₂-Z₃, wherein:
i) Z₁ is an insulin B-chain analog, comprising the amino acid sequence:
X₁X₂X₃QHLCGSHLVEALX₄LVCGERGFX₅YX₆X₇X₈X₉
wherein X₁ is F, Q or A; X₂ is V or G; X₃ is N, K, D, G, Q, A or E; X₄ is E, Y, Q, or H; X₅ is H or F; X₆ is G, T, S, H, V or is absent; X₇ is G, E, P, K, D, S, H or is absent; X₈ is G, E, K, P, Q, D, H or is absent; X₉ is G, T, S, E, K, A or is absent, provided that the insulin B-chain analog includes at least one modification from the amino acid sequence of the B-chain of a molecule of human insulin at X₄, X₅, X₆, X₇, X₈, or X₉ (SEQ ID NO:1);
ii) Z₂ is a first peptide linker comprising 5 to 10 amino acids, wherein at least 5 of said amino acids are G residues; and
iii) Z₃ is an insulin A-chain analog comprising the amino acid sequence:
GIVEQCCTSX₁CSLX₂QLENYCX₃X₄
wherein X₁ is T or I; X₂ is D, Y, Q or E; X₃ is G, N, S or A; and X₄ is any naturally occurring amino acid, or is absent, provided that if X₃ is N, then X₄ must be an amino acid other than G or N (SEQ ID NO:2);
b) a second peptide linker; and
c) a human IgG Fc region;
wherein the C-terminal residue of the insulin receptor agonist is directly fused to the N-terminal residue of the second peptide linker, and the C-terminal residue of the second peptide linker is directly fused to the N-terminal residue of the human IgG Fc region.

2. The fusion protein of claim 1, wherein:
the insulin B-chain analog includes at least one modification from the amino acid sequence of the human insulin B-chain at X₄ or X₅ of SEQ ID NO:1; and
and the insulin A-chain analog includes at least one modification from the amino acid sequence of the human insulin A-chain at X₁ or X₂ of SEQ ID NO:2.

3. The fusion protein of claim 2, wherein:
the insulin B-chain analog comprises the sequence of SEQ ID NO:1, wherein: X₁ is F;
X₂ is V; X₃ is N or D; X₄ is E; X₅ is H; and
the insulin A-chain analog comprises the sequence of SEQ ID NO:2, wherein: X₁ is I or T; X₂ is D; X₃ is G; and X₄ is absent.

4. The fusion protein of claim 3, wherein the insulin B-chain analog comprises the sequence of SEQ ID NO:1, wherein X₆-X₉ are each G.

5. The fusion protein of claim 1, wherein the first peptide linker comprises the following amino acid sequence:
X₁GX₂GGGG
wherein X₁ is G or is absent; and X₂ is G, S or is absent (SEQ ID NO:3).

6. The fusion protein of claim 5, wherein X₁ and X₂ of SEQ ID NO:3 are G and S, respectively.

7. The fusion protein of claim 1, wherein the insulin receptor agonist has the following amino acid sequence:
(SEQ ID NO: 5)
FVNQHLCGSHLVEALELVCGERGFHYGGGGGGSGGGGGIVEQCCTSTCSL
DQLENYCG.

8. The fusion protein of claim 1, wherein the second peptide linker is a peptide having between 10 and 25 amino acids, wherein at least 50% of said amino acids are G residues.

9. The fusion protein of claim 8, wherein the second peptide linker comprises a peptide having the sequence [GGGGX]ₙ
wherein X is Q, E or S; and wherein n is 2-5.

10. The fusion protein of claim 9, wherein the second peptide linker comprises the following amino acid sequence:
GGGGX₁GGGGX₂GGGGX₃GGGGX₄X₅X₆
X₁ is Q or E
X₂ is Q or E
X₃ is Q or E
X₄ is G, E, Q or is absent
X₅ is G or absent; and
X₆ is G or is absent
(SEQ ID NO:6).

11. The fusion protein of claim 1, wherein the second peptide linker has the following amino acid sequence:
(SEQ ID NO: 7)
GGGGQGGGGQGGGGQGGGGG.

12. The fusion protein of claim 1, wherein the human IgG Fc region is an Fc region from an IgG1, IgG2 or IgG4.

13. The fusion protein of claim 1, wherein the human IgG Fc region comprises an amino acid sequence selected from the group consisting of SEQ ID NO:8, SEQ ID NO:9 and SEQ ID NO:10.

14. A fusion protein having the amino acid sequence of SEQ ID NO:12.

15. A homodimer of two fusion proteins of claim 1.

16. A homodimer of two fusion proteins of claim 14.

17. A pharmaceutical composition comprising a fusion protein of claim 1 and further comprising one or more buffering agents, one or more surfactants, and one or more isotonicity agents.

18. The pharmaceutical composition of claim 17 wherein the pH ranges from about 5.5 to about 8.0.

19. The pharmaceutical composition of claim 17, further comprising an additional active ingredient.

20. The pharmaceutical composition of claim 19, wherein the additional active ingredient is a GLP-1R agonist.

21. The pharmaceutical composition of claim 20, wherein the GLP-1R agonist is dulaglutide.

22. A pharmaceutical composition comprising a homodimer of claim 16 and dulaglutide.

23. A method of treating a patient with diabetes mellitus comprising administering to a patient in need thereof a therapeutically effective amount of the fusion protein of claim 1.

24. The method of claim 23, wherein the fusion protein of claim 1 is administered in combination with a GLP-IR agonist.

25. The method of claim 24, wherein the GLP-1R agonist is dulaglutide.

26. A method of treating a patient with diabetes mellitus comprising administering to a patient in need thereof a homodimer of claim 16 in combination with dulaglutide.
